(19) United States
(12) Patent Application Publication
(10) Pub. No.: US 2006/0246575 A1
(43) Pub. Date: Nov. 2, 2006

(54) Microfluidic rare cell detection device

(75) Inventors: Christy A. Lancaster, Seattle, WA (US); C. Frederick Battrell, Redmond, WA (US); Jason Capodanno, Redmond, WA (US); John Gerdes, Columbine Valley, CO (US); Mark Kokoris, Bothell, WA (US); Melud Nabavi, Seattle, WA (US); Stephen Mordue, Seattle, WA (US); Robert McRuer, Mercer Island, WA (US); John Clemmens, Redmond, WA (US); Wayne L. Breidford, Seattle, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Micronics, Inc., Redmond, WA (US)

(21) Appl. No.: 11/331,587

(22) Filed: Jan. 13, 2006

Related U.S. Application Data
(60) Provisional application No. 60/643,833, filed on Jan. 13, 2005.

(51) Int. Cl. C12M 1/34

(52) U.S. Cl. 435/287.2; 435/288.5

(57) ABSTRACT

The present invention relates to microfluidic devices and methods for detecting rare cells. The disclosed microfluidic devices and methods integrate and automate sample preparation, cell labeling, cell sorting and enrichment, and DNA/RNA analysis of sorted cells.

### CROSS-REFERENCE TO RELATED APPLICATION

This application claims the benefit of U.S. Provisional Patent Application No. 60/643,833, filed Jan. 13, 2005, which application is hereby incorporated by reference in its entirety.

### BACKGROUND OF THE INVENTION

1. Field of the Invention

The present invention relates generally to microfluidic devices, and, more particularly, to microfluidic devices and methods for detecting rare cells.

2. Description of the Related Art

The biological changes that are now known to be associated with cancer cells encompass the full continuum from mutated or duplicated genomic sequences to shifts in gene expression patterns, as well as altered proteins. The challenge for practical transfer of the diversity of molecular information being generated in the characterization of cancer cells into routine clinical practice is the development of reproducible, integrated and automated methods for their measurement. One technical hurdle is defining a strategy for specimen analysis that includes the enrichment and detection of cancer cells, which are frequently found at low concentration in a high background of normal cells. In addition, maximum clinical utility would be enabled if the sorted cancer cells could be analyzed for protein, DNA, or mRNA expression alterations rapidly and from the same specimen. For example, detection of disseminated cancer cells in blood is one approach that is of particular importance. Unfortunately, current detection methods lack adequate sensitivity to reproducibly detect disseminated cancer cells, which can be as few as 1-10 cells per 10 ml of blood. Thus, there remains a need for more sensitive cancer cell detection methods that can be integrated into an automated analysis platform capable of confirming protein, DNA, or mRNA alterations. The present invention addresses this need and provides further related advantages.

One of the current approaches to enriching for rare cancer cells in biological samples, such as blood, is flow cytometry. Presently, the state of the art in flow cytometry and cell sorting technology uses a hydrodynamically focused core stream, which is focused in two dimensions to roughly the size of a single cell (˜10 microns) in the dimensions orthogonal to flow. This produces a single file cell stream, which can be presented to a light scatter, fluorescent detector, or image-based cell detector system. However, this scheme suffers from a significant limitation—that the detectors may only detect and direct one cell at a time. Accordingly, in order to process large quantities of cells, the fluidic systems must be run very fast past the detectors. For example, speeds from one to forty meters per second past the detector may be necessary for some applications. Following detection, cells are then partitioned into micro droplets and each micro droplet is charged so that it may be electrostatically deflected into separate bins for sorting. Unfortunately, due to the technically complex methodology and result interpretation involved in current flow cytometry methods, such analyses are generally performed by reference laboratories. Thus, there remains a need for detection methods compatible with routine clinical practice. The present invention addresses this need and provides further related advantages.

Microfluidic devices have become popular in recent years for performing analytical testing. Using tools developed by the semiconductor industry to miniaturize electronics, it has become possible to fabricate intricate fluid systems which can be inexpensively mass produced. Systems have been developed to perform a variety of analytical techniques for the acquisition and processing of information. The ability to perform analyses microfluidically provides substantial advantages of throughput, reagent consumption, and automatability. Another advantage of microfluidic systems is the ability to integrate a plurality of different operations in a single “lap-on-a-chip” device for performing processing of reactants for analysis and/or synthesis.

Microfluidic devices may be constructed in a multi-layer laminated structure wherein each layer has channels and structures fabricated from a laminate material to form microscale voids or channels where fluids flow. A microscale or microfluidic channel is generally defined as a fluid passage which has at least one internal cross-sectional dimension that is less than 500 μm and typically between about 0.1 μm and about 500 μm.

U.S. Pat. No. 5,716,852, which patent is hereby incorporated by reference in its entirety, is an example of a microfluidic device. The '852 patent teaches a microfluidic system for detecting the presence of analyte particles in a sample stream using a laminar flow channel having at least two input channels which provide an indicator stream and a sample stream, where the laminar flow channel has a depth sufficiently small to allow laminar flow of the streams and length sufficient to allow diffusion of particles of the analyte into the indicator stream to form a detection area, and having an outlet out of the channel to form a single mixed stream. This device, which is known as a T-Sensor, allows the movement of different fluidic layers next to each other within a channel without mixing other than by diffusion. A sample stream, such as whole blood, a receptor stream, such as an indicator solution, and a reference stream, which may be a known analyte standard, are introduced into a common microfluidic channel within the T-Sensor, and the streams flow next to each other until they exit the channel. Smaller particles, such as ions or small proteins, diffuse rapidly across the fluid boundaries, whereas larger molecules diffuse more slowly. Large particles, such as blood cells, show no significant diffusion within the time the two flow streams are in contact.

Typically, microfluidic systems require some type of external fluidic driver to function, such as piezoelectric pumps, micro-syringe pumps, electroosmotic pumps, and the like. However, in U.S. patent application Ser. No. 09/684,094, which application is assigned to the assignee of the present invention and is hereby incorporated by reference in its entirety, microfluidic systems are described which are completely driven by inherently available internal forces such as gravity, hydrostatic pressure, capillary force, absorption by porous material or chemically induced pressures or vacuums.

In addition, many different types of valves for use in controlling fluids in microscale devices have been developed. For example, U.S. Pat. No. 6,432,212 describes one-way valves (also known as check valves) for use in laminated microfluidic structures, U.S. Pat. No. 6,581,899 describes ball bearing valves for use in laminated microfluidic structures, U.S. patent application Ser. No. 10/960,890, which application is assigned to the assignee of the present invention, describes a pneumatic valve interface, also known as a zero dead volume valve or passive valve, for use in laminated microfluidic structures, and U.S. Provisional Patent Application entitled “Electromagnetic Valve Interface for Use in Microfluidic Structures”, filed on Jan. 13, 2006 and assigned to the assignee of the present invention, describes an electromagnetically actuated valve interface for use in laminated microfluidic structures. The foregoing patents and patent applications are hereby incorporated by reference in their entirety.

Although there have been many advances in the field, there remains a need for new and improved microfluidic devices for manipulating and analyzing fluid samples. In particular, there remains a need for microfluidic devices incorporating a plurality of sample preparation and analysis techniques, such as a microfluidic device for detecting rare cells. The present invention addresses these needs and provides further related advantages.

### BRIEF SUMMARY OF THE INVENTION

In brief, the present invention relates to microfluidic devices and methods for detecting rare cells. The disclosed devices and methods integrate and automate sample preparation, cell labeling, cell sorting and enrichment, and DNA/RNA analysis of sorted cells.

In one embodiment, a microfluidic device for detecting rare cells is provided that comprises: (1) means for introducing a biological sample into the microfluidic device, wherein the biological sample comprises one or more labeled cells; (2) means for sheathing the biological sample with a buffer liquid to form a thin ribbon of the biological sample; (3) means for facilitating the detection of the labeled cells in the biological sample; (4) means for separating the labeled cells from the biological sample; (5) means for lysing the labeled cells; (6) means for collecting RNA and DNA released from the lysed labeled cells; and (7) means for performing quantitative PCR analysis of the collected RNA and DNA.

In a more specific embodiment, the means for introducing a biological sample into the microfluidic device comprises a sample inlet port fluidly connected to a sample inlet microfluidic channel.

In another more specific embodiment, the means for sheathing the biological sample with a buffer liquid to form a thin ribbon of the biological sample comprises a thin ribbon sheath flow assembly. The thin ribbon sheath flow assembly may comprise a sample microfluidic channel, a first sheath liquid microfluidic channel and a second sheath liquid microfluidic channel, wherein the first and second sheath liquid microfluidic channels are positioned on opposing sides of, and fluidly converge with, the sample microfluidic channel.

In another more specific embodiment, the means for facilitating the detection of the labeled cells in the biological sample comprises an optical viewing window positioned over a portion of a sheathed sample microfluidic channel.

In another more specific embodiment, the means for separating the labeled cells from the biological sample comprises a cell sorting slit structure.

In another more specific embodiment, the means for separating the labeled cells from the biological sample comprises a cell sorting flexible film structure comprising a flexible film membrane, the flexible film membrane being deformable into a sheathed sample microfluidic channel upon the application of pneumatic pressure.

In another more specific embodiment, the means for separating the labeled cells from the biological sample comprises an electromagnetically actuated valve. The electromagnetically actuated valve may comprise a metal foil.

In another more specific embodiment, the means for lysing the labeled cells comprises a first membrane, adapted to capture the labeled cells, and a lysis buffer microfluidic channel fluidly connected to the first membrane. The first membrane may be a polybutylene terephthalate (PBT) membrane, such as a Lukesorb® membrane.

In another more specific embodiment, the means for lysing the labeled cells comprises a lysis buffer sheath flow assembly. The lysis buffer sheath flow assembly may comprise a sorted cell microfluidic channel, a first lysis buffer microfluidic channel and a second lysis buffer microfluidic channel, wherein the first and second lysis buffer microfluidic channels are positioned on opposing sides of, and fluidly converge with, the sorted cell microfluidic channel.

In another more specific embodiment, the means for collecting RNA and DNA released from the lysed labeled cells comprises a second membrane, adapted to capture the released RNA and DNA. The second membrane may comprise glass or silicate.

In another more specific embodiment, the means for performing quantitative PCR analysis of the collected RNA and DNA comprises a PCR amplification chamber. The PCR amplification chamber may comprise PCR probe and primer reagents pre-loaded or printed into the PCR amplification chamber.

In another more specific embodiment, the biological sample is a blood sample.

In a second embodiment, a microfluidic device for detecting rare cells is provided that comprises: (1) means for introducing a biological sample into the microfluidic device; (2) means for sheathing the biological sample with a labeling buffer liquid to form a thin ribbon of the biological sample and label one or more cells in the biological sample; (3) means for facilitating the detection of the labeled cells in the biological sample; (4) means for separating the labeled cells from the biological sample; (5) means for lysing the labeled cells; (6) means for collecting RNA and DNA released from the lysed labeled cells; and (7) means for performing quantitative PCR analysis of the collected RNA and DNA.

In a more specific embodiment, the means for introducing a biological sample into the microfluidic device comprises a sample inlet port fluidly connected to a sample inlet microfluidic channel.

In another more specific embodiment, the means for sheathing the biological sample with a labeling buffer liquid to form a thin ribbon of the biological sample and label one or more cells in the biological sample comprises a thin ribbon labeling sheath flow assembly. The thin ribbon labeling sheath flow assembly may comprise a sample microfluidic channel, a first labeling sheath liquid microfluidic channel and a second labeling sheath liquid microfluidic channel, wherein the first and second labeling sheath liquid microfluidic channels are positioned on opposing sides of, and fluidly converge with, the sample microfluidic channel.

In another more specific embodiment, the means for facilitating the detection of the labeled cells in the biological sample comprises an optical viewing window positioned over a portion of a sheathed sample microfluidic channel.

In another more specific embodiment, the means for separating the labeled cells from the biological sample comprises a cell sorting slit structure.

In another more specific embodiment, the means for separating the labeled cells from the biological sample comprises a cell sorting flexible film structure comprising a flexible film membrane, the flexible film membrane being deformable into a sheathed sample microfluidic channel upon the application of pneumatic pressure.

In another more specific embodiment, the means for separating the labeled cells from the biological sample comprises an electromagnetically actuated valve. The electromagnetically actuated valve may comprise a metal foil.

In another more specific embodiment, the means for lysing the labeled cells comprises a first membrane, adapted to capture the labeled cells, and a lysis buffer microfluidic channel fluidly connected to the first membrane. The first membrane may be a polybutylene terephthalate (PBT) membrane, such as a Lukesorb® membrane.

In another more specific embodiment, the means for lysing the labeled cells comprises a lysis buffer sheath flow assembly. The lysis buffer sheath flow assembly may comprise a sorted cell microfluidic channel, a first lysis buffer microfluidic channel and a second lysis buffer microfluidic channel, wherein the first and second lysis buffer microfluidic channels are positioned on opposing sides of, and fluidly converge with, the sorted cell microfluidic channel.

In another more specific embodiment, the means for collecting RNA and DNA released from the lysed labeled cells comprises a second membrane, adapted to capture the released RNA and DNA. The second membrane may comprise glass or silicate.

In another more specific embodiment, the means for performing quantitative PCR analysis of the collected RNA and DNA comprises a PCR amplification chamber. The PCR amplification chamber may comprise PCR probe and primer reagents pre-loaded or printed into the PCR amplification chamber.

In another more specific embodiment, the biological sample is a blood sample.

These and other aspects of the invention will be apparent upon reference to the attached figures and following detailed description.

### BRIEF DESCRIPTION OF THE SEVERAL VIEWS OF THE DRAWINGS

FIG. 1 is a flow chart showing the steps in a representative method for detecting rare cells in accordance with aspects of the present invention.

FIG. 2 is a schematic diagram of a representative microfluidic device for detecting rare cells in accordance with aspects of the present invention.

FIGS. 3A-3C are a series of cross-sectional views of a microfluidic device illustrating the operation of a representative sub-circuit for antibody labeling of white blood cells in accordance with aspects of the present invention.

FIGS. 4A-4D are a series of cross-sectional views of a microfluidic device illustrating the operation of a representative sub-circuit for both antibody labeling of white blood cells and lysing of red blood cells in accordance with aspects of the present invention.

FIGS. 5A-5I are a number of cross-sectional views of various microfluidic devices and structures illustrating the operation of various representative sub-circuits for sorting antibody labeled cells in accordance with aspects of the present invention.

FIGS. 6A-6D are a series of cross-sectional views of a microfluidic device illustrating the operation of a representative sub-circuit for white blood cell capture and lysis in accordance with aspects of the present invention.

FIGS. 7A-7F are a series of cross-sectional views of a microfluidic device illustrating the operation of a representative sub-circuit for nucleic acid capture and purification in accordance with aspects of the present invention.

FIGS. 8A-8B are cross-sectional views of a microfluidic device illustrating the operation of a representative sub-circuit for nucleic acid amplification in accordance with aspects of the present invention.

FIG. 8C is a photograph of a representative system incorporating the microfluidic device of FIGS. 8A-8B for nucleic acid amplification in accordance with aspects of the present invention.

FIGS. 9A-9B are cross-sectional views of a representative microfluidic device incorporating the sub-circuits of FIGS. 6A-6D, 7A-7F and 8A-8B in accordance with aspects of the present invention.

FIG. 10 shows the results of the LightCycler assays of Example 2.

### DETAILED DESCRIPTION OF THE INVENTION

As noted previously, the present invention relates to microfluidic devices and methods for detecting rare cells. The devices of the present invention utilize a plurality of microfluidic channels, inlets, valves, membranes, pumps, liquid barriers and other elements arranged in various configurations to manipulate the flow of a fluid sample in order to prepare such sample for analysis and to analyze the fluid sample. In the following description, certain specific embodiments of the present devices and methods are set forth, however, persons skilled in the art will understand that the various embodiments and elements described below may be combined or modified without deviating from the spirit and scope of the invention.

As one of ordinary skill in the art will appreciate, the term “rare cell” used herein refers to uniquely identifiable cells that occur in a sample (such as a biological sample) in extremely low concentrations (i.e., on the order of one in millions) and may be associated with a number of conditions including cancer.

In addition, as one of ordinary skill in the art will appreciate, the term “biological sample” used herein includes (but is not limited to) liquid biological samples such as blood samples, urine samples, and semen samples. For purposes of illustration, the following description frequently refers to “blood samples,” however, as one of ordinary skill in the art will appreciate, the disclosure and described embodiments equally apply to, and encompass, other liquid biological samples, such as urine and semen.

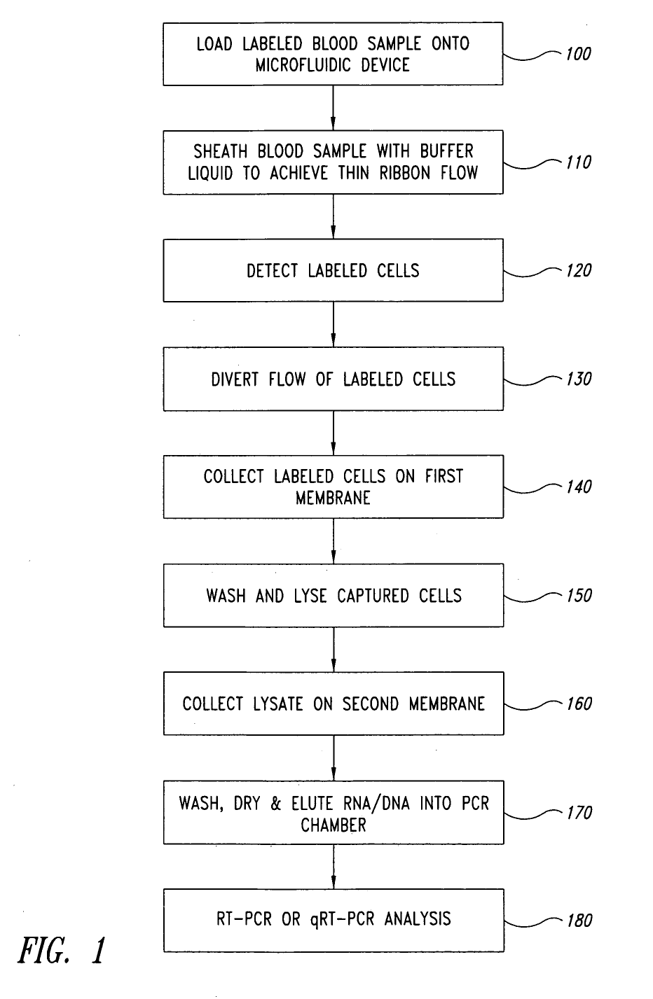

FIG. 1 is a flow chart showing the steps in a representative method for detecting rare cells in accordance with aspects of the present invention. Such a method comprises the following steps: (1) loading a blood sample containing one or more labeled cells onto a microfluidic device (indicated by reference number 100 in FIG. 1); (2) sheathing the blood sample with a buffer liquid to achieve a thin ribbon (i.e., one cell layer thick) flow of the blood sample between two streams of the buffer liquid (indicated by reference number 110 in FIG. 1); (3) detecting the labeled cells in the blood sample (indicated by reference number 120 in FIG. 1); (4) diverting the flow of a portion of the blood sample containing the labeled cells, thereby separating the labeled cells from the bulk of the blood sample (indicated by reference number 130 in FIG. 1); (5) collecting the labeled cells on a first membrane (e.g., a polybutylene terephthalate (PBT) membrane, such as a Lukesorb® membrane) (indicated by reference number 140 in FIG. 1); (6) washing and lysing the collected labeled cells on the first membrane (indicated by reference number 150 in FIG. 1); (7) collecting the lysate (which contains RNA and DNA released from the lysed labeled cells) on a second membrane (e.g., glass or silicate) (indicated by reference number 160 in FIG. 1); (8) washing and drying the lysate on the second membrane (indicated by reference number 170 in FIG. 1); (9) eluting the RNA and DNA collected on the second membrane into a PCR chamber (indicated by reference number 170 in FIG. 1); and (10) performing quantitative PCR (i.e., polymerase chain reaction) or qRT-PCR (quantitative reverse transcription polymerase chain reaction) analysis of the collected RNA and DNA (indicated by reference number 180 in FIG. 1).

In more specific embodiments of the foregoing method, and as described in more detail below: (1) certain cells in the blood sample may be labeled with a fluorescently labeled monoclonal antibody (e.g., CD-34) that binds to a specific antigen found on the surface of rare cells in the blood sample; and (2) the fluorescently labeled cells may be detected by an optical device through an optical viewing window or area of the microfluidic device. Quantitative PCR (i.e., polymerase chain reaction) and qRT-PCR (quantitative reverse transcription polymerase chain reaction) analyses may be performed on the microfluidic device by incorporating the requisite probes and primers for PCR into the microfluidic device (either in liquid (i.e., blister pouches) or dried (i.e., printed) form), incorporating a PCR amplification chamber in the microfluidic device and interfacing the microfluidic device with thermal cycling heating devices, such as Peltier devices. Representative microfluidic devices having integrated heat cycling systems are described in U.S. patent application Ser. No. 10/862,826, which application is assigned to the assignee of the present invention and is hereby incorporated by reference in its entirety. In addition, software may be utilized to control and perform each step in the method and algorithms may be devised to permit the device to report the number of labeled cells in a given volume of a whole blood sample as well as the number of genetic labeled cells of interest in the same blood sample.

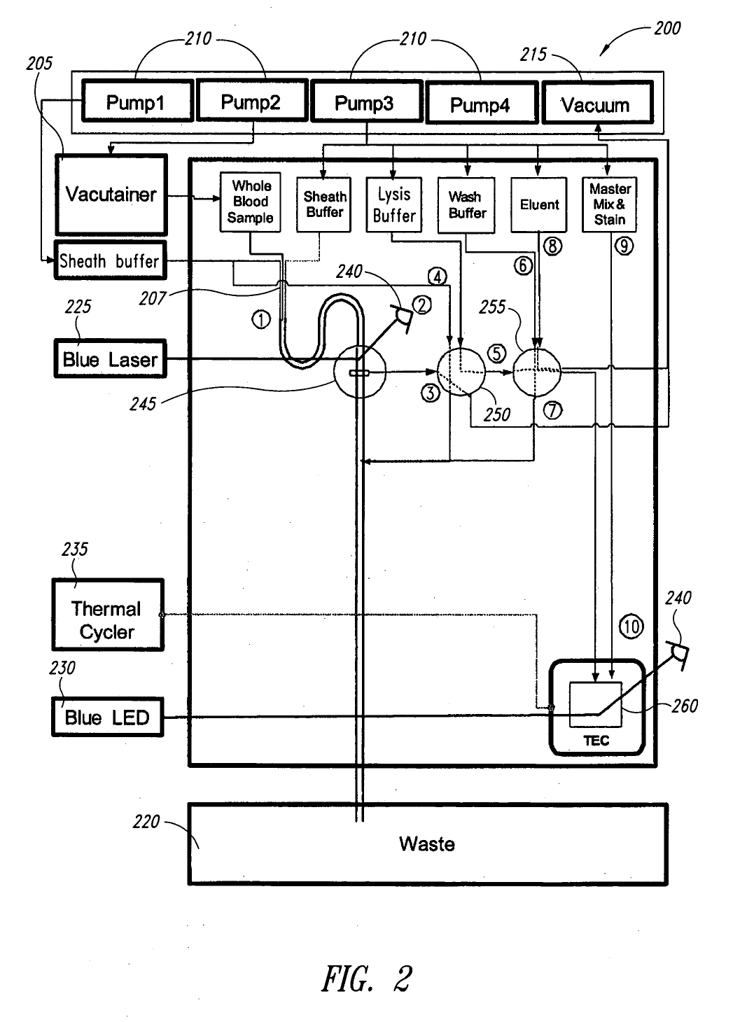

FIG. 2 is a schematic diagram of a representative microfluidic device 200 for detecting rare cells in accordance with aspects of the present invention. As shown, a vacutainer 205, containing a dried fluorescently labeled antibody, is filled with a whole blood sample (˜1-10 mL). The vacutainer 205 is then mated to the microfluidic device 200 and the assembly is inserted into a pumping/reading device or station (not specifically identified). The pumping/reading device comprises a plurality of micropumps 210, a vacuum line 215, a waste line 220, a blue laser 225, a blue LED 230, a thermal cycler 235 and several detectors 240, as well as reservoirs for wash fluids and waste fluids.

In operation, the pumping/reading device pumps the whole blood sample from the vacutainer 205 into a sample inlet microfluidic channel (not specifically shown) of the microfluidic device 200 via a sample inlet port (not specifically shown) and through a thin ribbon sheath flow assembly 207 on the microfluidic device 200, thereby causing the blood sample to be sheathed in a buffer liquid to create a thin ribbon flow of the blood sample (indicated by label 1 in FIG. 2). The thin ribbon flow of the blood sample proceeds to an optical viewing window 245, where it is illuminated with a blue laser 225 and a detector 240 monitors for labeled cells (indicated by label 2 in FIG. 2). When a labeled cell is detected, a small volume of the blood sample containing the cell is diverted onto a first membrane 250 (indicated by label 3 in FIG. 2). In this way, multiple cells are viewed and sorted, not individually, but as a whole cell row or section of the ribbon at a time. When a sufficient number of labeled cells have been captured, or when the blood sample is depleted, the first membrane 250 is washed to remove unwanted cells (indicated by label 4 in FIG. 2). A lysis buffer is then passed over the first membrane 250, lysing the labeled cells and releasing a lysate comprising RNA and DNA, which is captured on a second membrane 255 (indicated by label 5 in FIG. 2). The second membrane 255 is then washed (indicated by label 6 in FIG. 2), dried (indicated by label 7 in FIG. 2) and the RNA/DNA is eluted (indicated by label 8 in FIG. 2) into a PCR amplification chamber 260. The collected RNA/DNA then is mixed with reagents allowing quantitative PCR as well as qRT-PCR to go forward (indicated by label 9 in FIG. 2). In qRT-PCR, a blue LED 230 illuminates the sample each cycle in order to detect the increase in fluorescence per cycle (indicated by label 10 in FIG. 2).

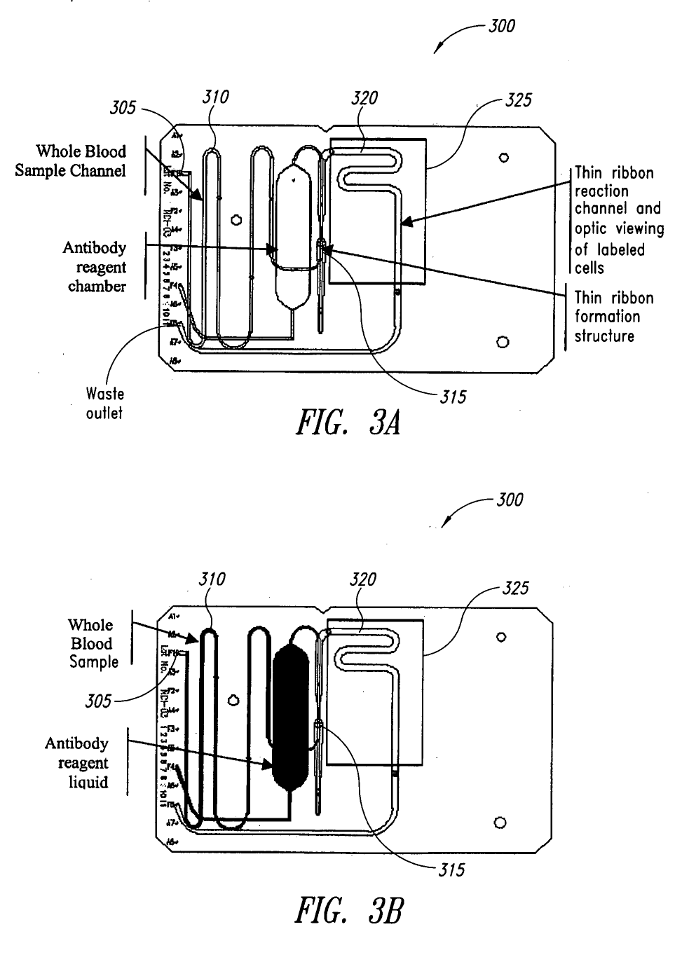

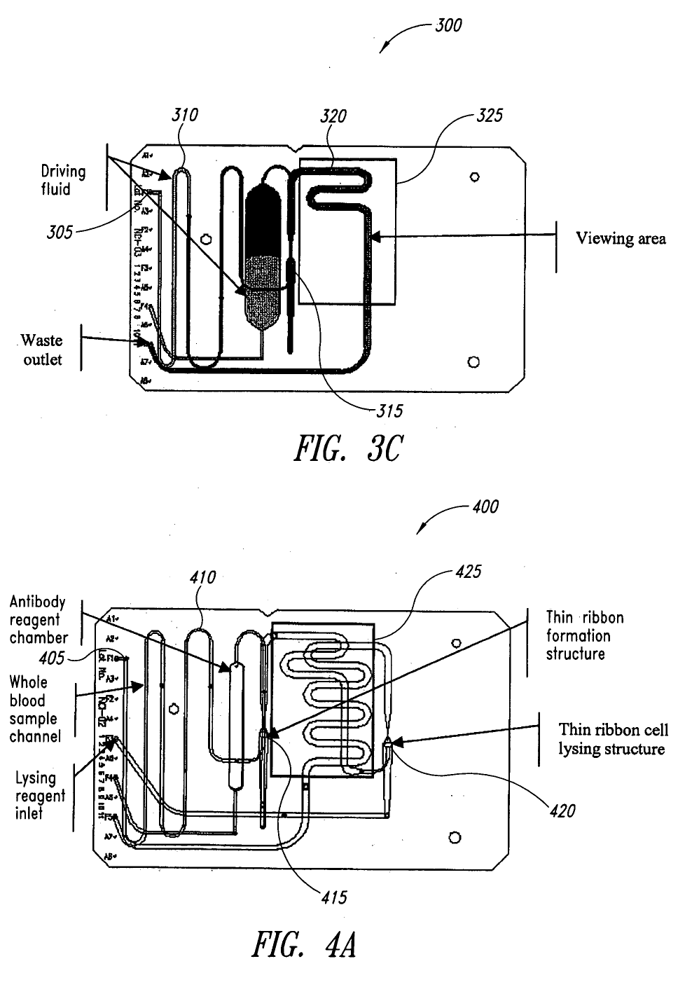

As described above, the embodiments of FIGS. 1 and 2 provide for a pre-labeled blood sample to be introduced into a microfluidic device. However, as one of ordinary skill in the art will appreciate, in alternate embodiments, the microfluidic device may be configured to also provide for the labeling of the blood sample. For example, FIGS. 3A-3C are a series of cross-sectional views of a microfluidic device 300 illustrating, for example, the operation of a representative sub-circuit for antibody labeling of white blood cells in accordance with aspects of the present invention. As shown, both a whole blood sample and a labeling buffer liquid (e.g., an antibody reagent) are loaded onto a microfluidic device 300. The blood sample is introduced into the microfluidic device 300 through a sample inlet port 305 and a sample inlet microfluidic channel 310. In the illustrated embodiment, a driving fluid is utilized to push the blood sample and the antibody reagent through a thin ribbon sheath flow assembly 315 (more specifically, a thin ribbon labeling sheath flow assembly 315 in this embodiment) to form a thin ribbon of the blood sample between streams of the antibody reagent. As noted above, the thin ribbon sheath flow assembly 315 may comprise a first sheath liquid microfluidic channel and a second sheath liquid microfluidic channel, wherein the first and second sheath liquid microfluidic channels are positioned on opposing sides of, and fluidly converge with, the sample microfluidic channel. U.S. Pat. No. 6,576,194, which patent is incorporated herein by reference in its entirety, further describes such a sheath flow assembly. While in this thin ribbon formation, and while flowing through a sheathed sample microfluidic channel 320, diffusion between the fluid streams facilitates the labeling of white blood cells in the blood sample with the antibody. The labeled cells may then be optically detected through the indicated optical viewing window 325, which is positioned over a portion of the sheathed sample microfluidic channel 320.

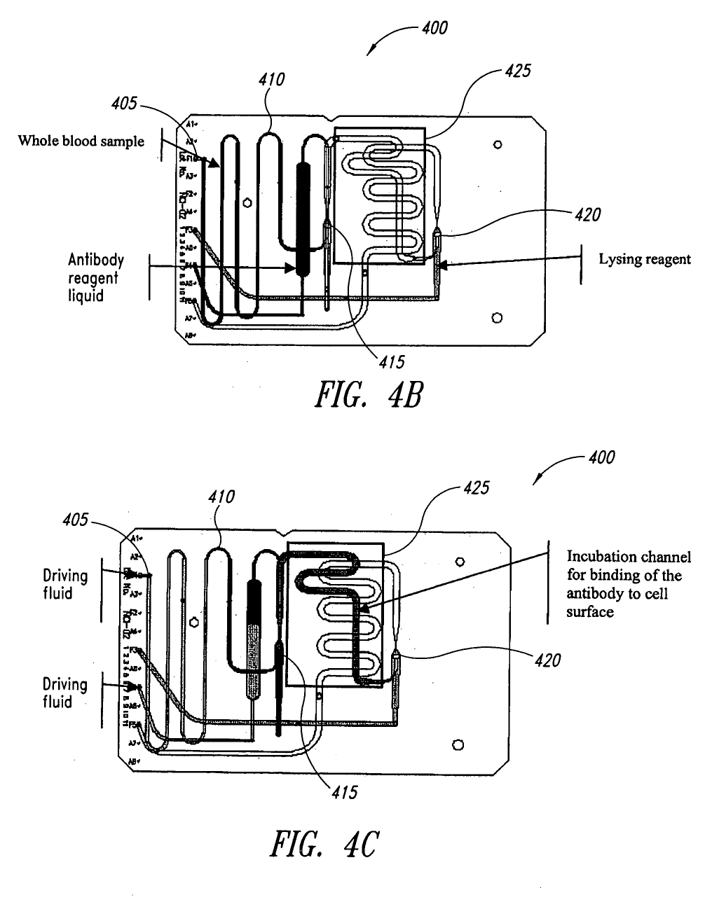

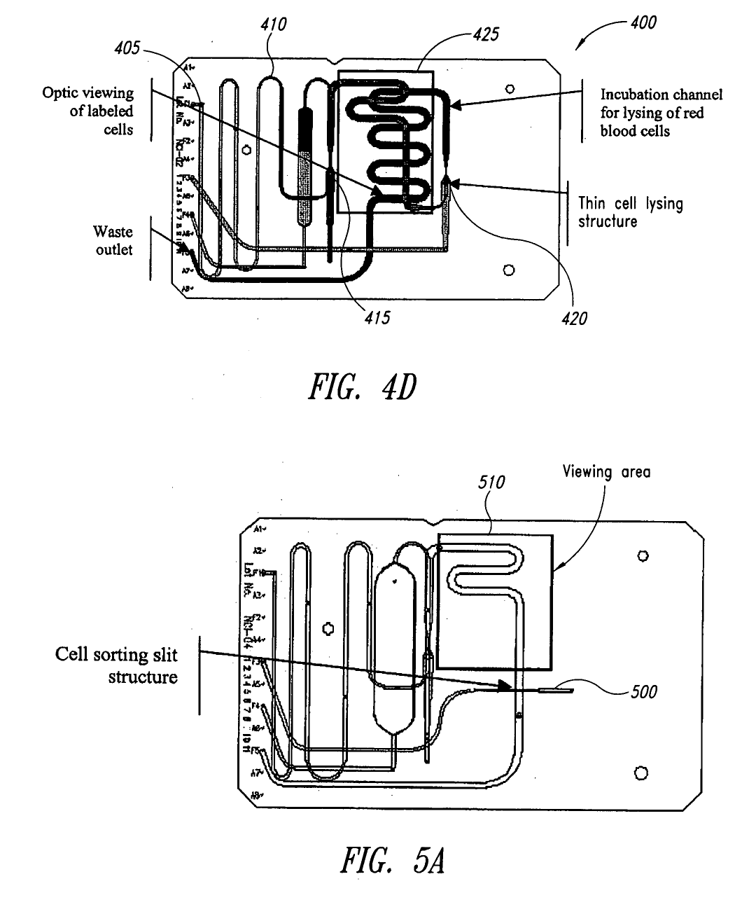

Another embodiment that provides for labeling of the blood sample in the microfluidic device is illustrated in FIGS. 4A-4D, which show a series of cross-sectional views of a microfluidic device 400 illustrating, for example, the operation of a representative sub-circuit for both antibody labeling of white blood cells and lysing of red blood cells in accordance with aspects of the present invention. As in FIGS. 3A-3C, both a whole blood sample and a labeling buffer liquid (i.e., antibody reagent liquid) are loaded onto the microfluidic device 400. The blood sample is introduced into the microfluidic device 400 through a sample inlet port 405 and a sample inlet microfluidic channel 410. A driving fluid is utilized to push the blood sample and the antibody reagent through a thin ribbon sheath flow assembly 415 (more specifically, a thin ribbon labeling sheath flow assembly in the illustrated embodiment) to form a thin ribbon of the blood sample between streams of the antibody reagent. As noted above, the thin ribbon sheath flow assembly 415 may comprise a first sheath liquid microfluidic channel and a second sheath liquid microfluidic channel, wherein the first and second sheath liquid microfluidic channels are positioned on opposing sides of, and fluidly converge with, the sample microfluidic channel. U.S. Pat. No. 6,576,194, which patent is incorporated herein by reference in its entirety, further describes such a sheath flow assembly. However, in the device of FIGS. 4A-4D, a lysing reagent liquid is also loaded onto the microfluidic device 400 and, following the labeling of the white blood cells in the blood sample with the antibody, the driving fluid is utilized to push the lysing reagent and labeled blood sample through a lysis buffer sheath flow assembly 420 to produce laminar flow of such fluids. Similar to the thin ribbon sheath flow assembly, the lysis buffer sheath flow assembly may comprise a first lysis buffer microfluidic channel and a second lysis buffer microfluidic channel, wherein the first and second lysis buffer microfluidic channels are positioned on opposing sides of, and fluidly converge with, the sample microfluidic channel. Diffusion between the resulting fluid streams results in the lysing of red blood cells in the blood sample. As a result, the remaining labeled white blood cells may be more easily detected through the indicated optical viewing window 425. In other embodiments, the lysis buffer sheath flow assembly 420 may be positioned downstream of a means for separating the labeled cells from the blood sample.

Further examples of microfluidic devices that provide for hydrodynamic focusing and lysing cells are described in U.S. Pat. No. 6,674,525, which patent is assigned to the assignee of the present invention and is hereby incorporated by reference in its entirety.

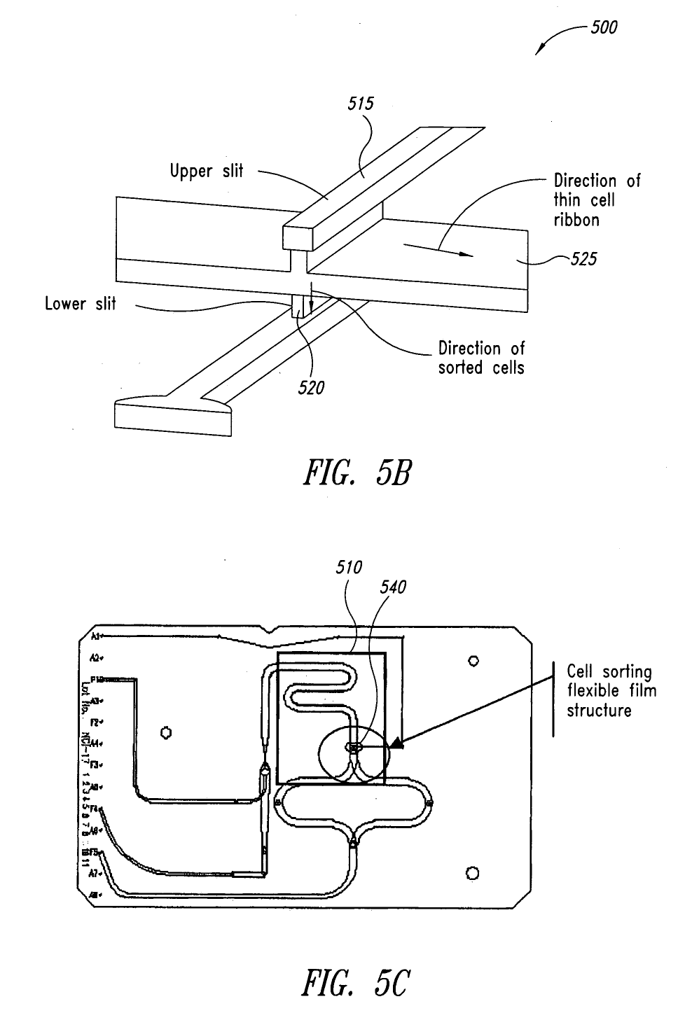

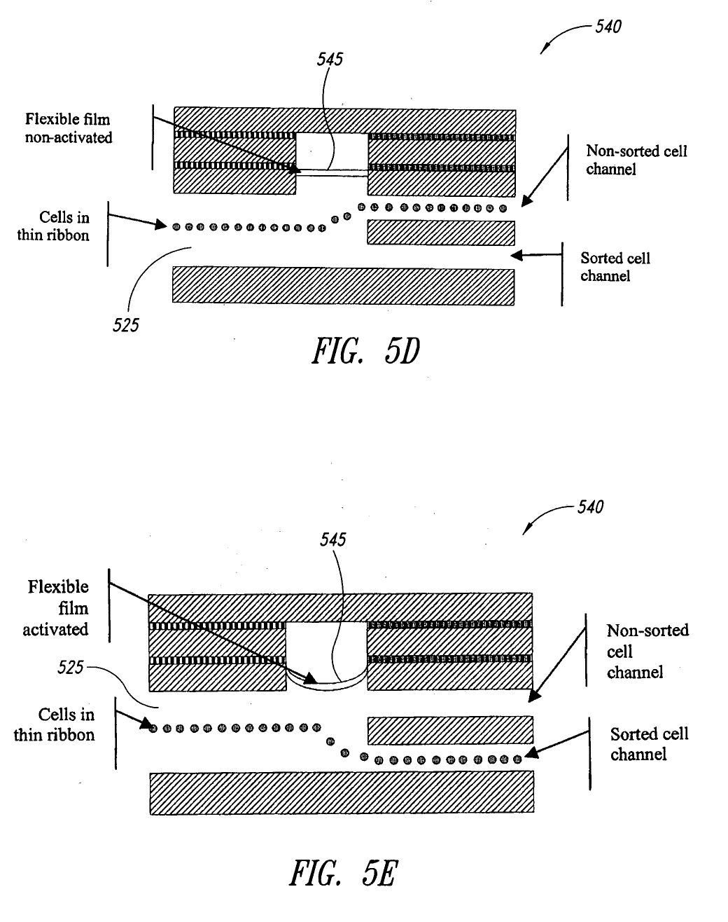

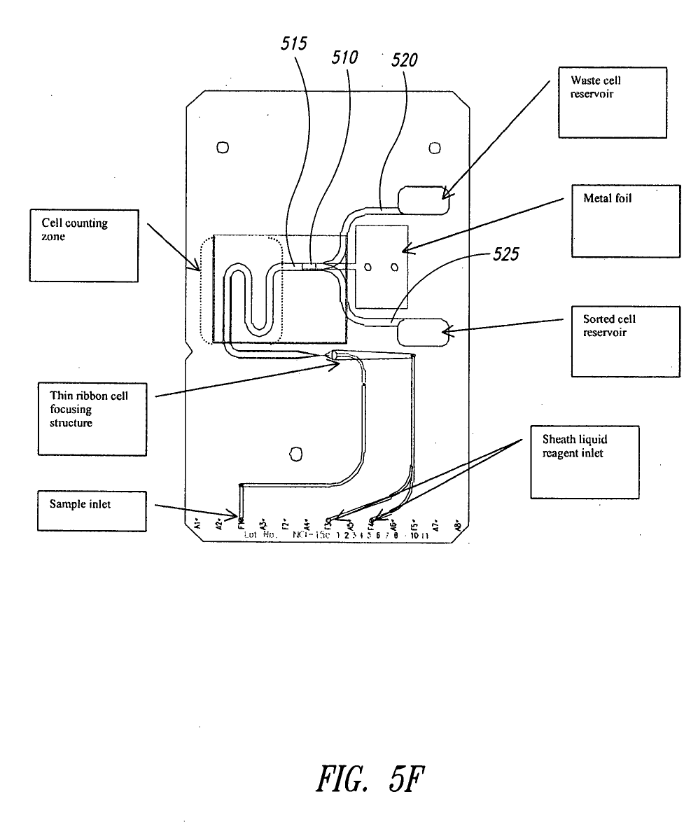

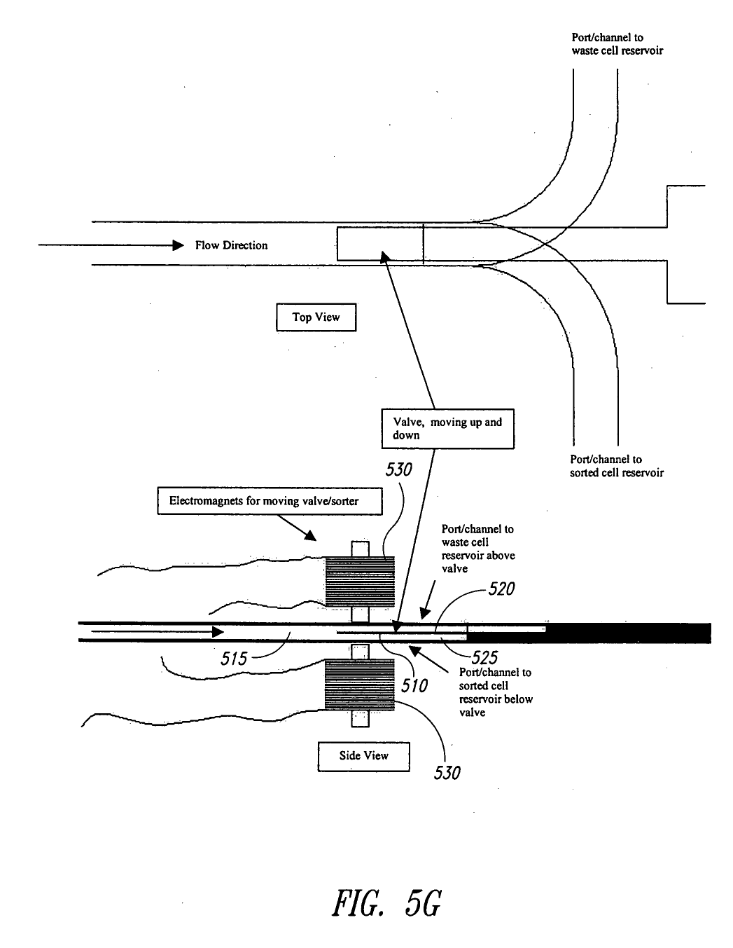

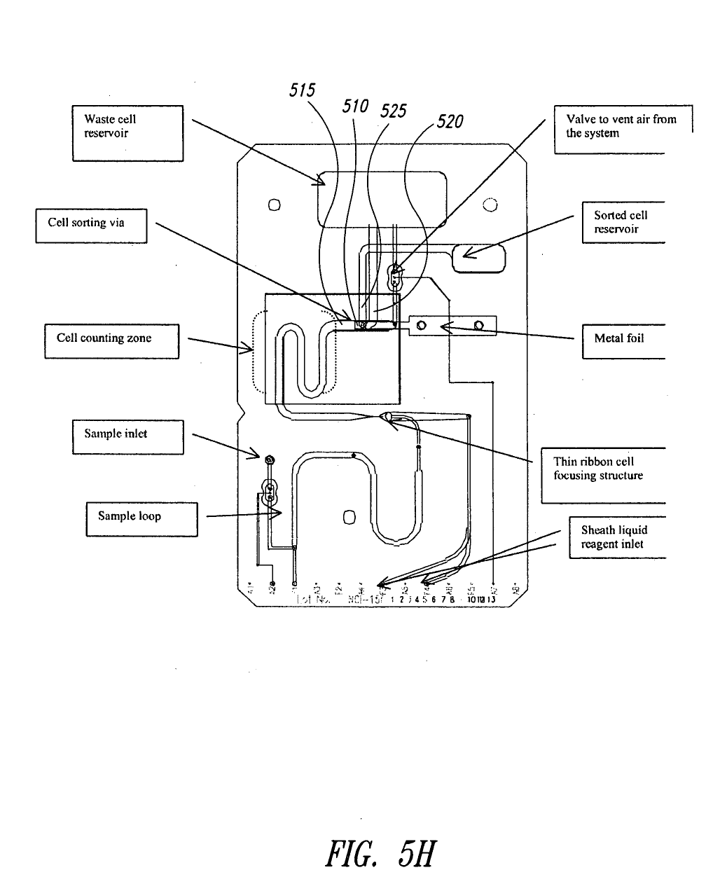

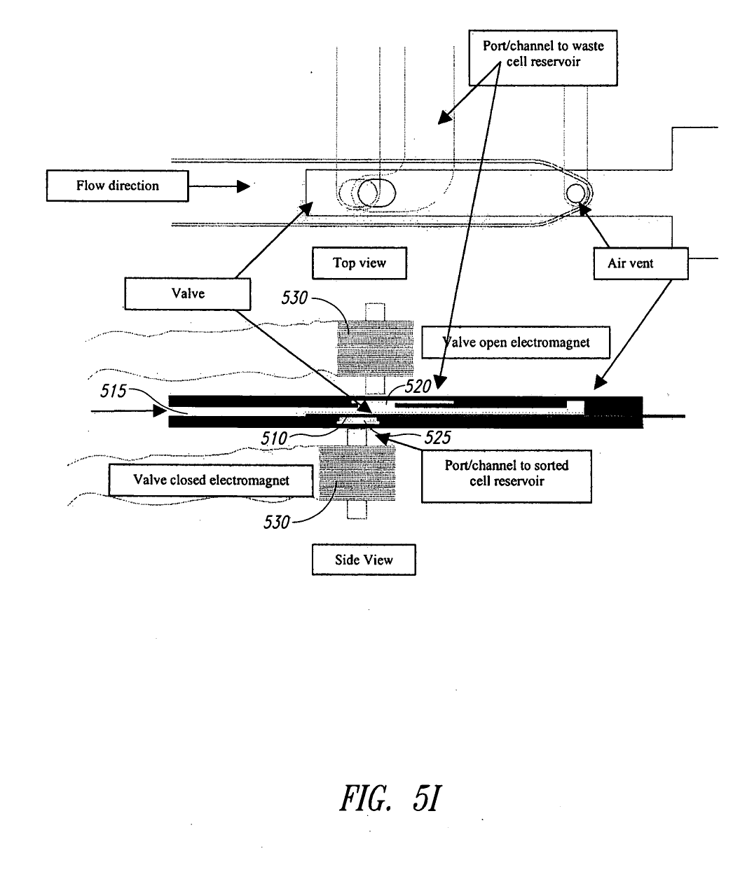

As described with respect to FIGS. 1 and 2, following the detection of a labeled cell, a small volume of the whole blood sample containing the cell is diverted. As one of skill in the art will appreciate, a wide range of microfluidic channels, valves, membranes, pumps, liquid barriers and other elements may be arranged in various configurations to achieve this result. For example, FIGS. 5A-5G show a number of cross-sectional views of various microfluidic devices and structures illustrating the operation of various representative sub-circuits for sorting antibody labeled cells in accordance with aspects of the present invention. Further examples of microfluidic devices for hydrodynamically focusing and sorting cells are described in U.S. Patent Application Publication No. 2003/0175980, which application is assigned to the assignee of the present invention and is hereby incorporated by reference in its entirety.

In one embodiment, shown in FIGS. 5A-5B, a representative sub-circuit for sorting labeled cells comprises a cell sorting slit structure 500 positioned downstream of the optical viewing window 510. The cell sorting slit structure 500 comprises both an upper slit 515 and a lower slit 520 positioned perpendicular to the primary sample microfluidic channel 525. Pulses of pneumatic pressure through the upper and lower slits, 515 and 520, respectively, may be utilized to divert the flow of the blood sample. For example, a pressure pulse through the upper slit 515 may be utilized to divert the flow from the primary sample microfluidic channel 525 to the lower slit 520. The width of the upper and lower slits 515 and 520, respectively, may be on the order of 25-200 microns.

In another embodiment, shown in FIGS. 5C-5E, a representative sub-circuit for sorting labeled cells comprises a cell sorting flexible film structure 540 positioned downstream of the optical viewing window 510. The cell sorting flexible film structure 540 comprises a flexible film membrane 545 that may be deformed into the primary microfluidic channel 525 by the application of pneumatic pressure. As more specifically shown in FIGS. 5D-5E, by deforming the flexible film membrane 545 in this manner, the flow of the blood sample may be diverted from the primary sample microfluidic channel 525.

In another embodiment, shown in FIGS. 5F-5I, a representative sub-circuit for sorting labeled cells comprises an electromagnetically actuated valve 510. In one embodiment, shown in FIGS. 5F-5G, the electromagnetically actuated valve 510 comprises a metal foil disposed between two laminate layers of the device and having one end “floating” in the primary microfluidic channel 515. By alternately actuating the “off-card” electromagnets 530 which are interfaced with the device, the metal foil 510 may be utilized to divert the flow of the blood sample between the two microfluidic channels 520 and 525 downstream of the metal foil 510 (in the illustrated embodiment, one of the channels 520 leads to a waste cell reservoir, and the other of the channels 525 leads to a sorted cell reservoir). In another embodiment, shown in FIGS. 5H-5I, the electromagnetically actuated valve 510 comprises a metal foil disposed between two laminate layers of the device and having one end normally disposed against the one surface (e.g., the bottom surface) of the primary microfluidic channel 515. As in the embodiment of FIGS. 5F-5G, by alternately actuating the “off-card” electromagnets 530 which are interfaced with the device, the metal foil 510 may be utilized to divert the flow of the blood sample between the two microfluidic channels 520 and 525 downstream of the metal foil 510 (in the illustrated embodiment, one of the channels 520 leads to a waste cell reservoir, and the other of the channels 525 leads to a sorted cell reservoir). These representative electromagnetically actuated valves are further described in U.S. Provisional Patent Application entitled “Electromagnetic Valve Interface for Use in Microfluidic Structures”, filed on Jan. 13, 2006 and assigned to the assignee of the present invention, which application is hereby incorporated herein by reference in its entirety.

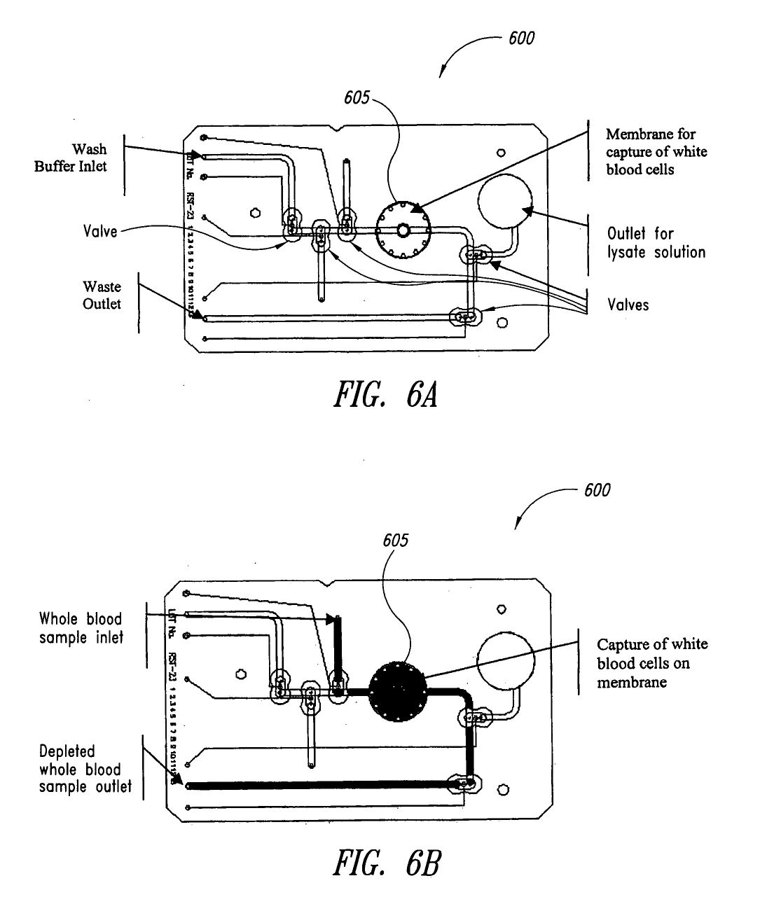

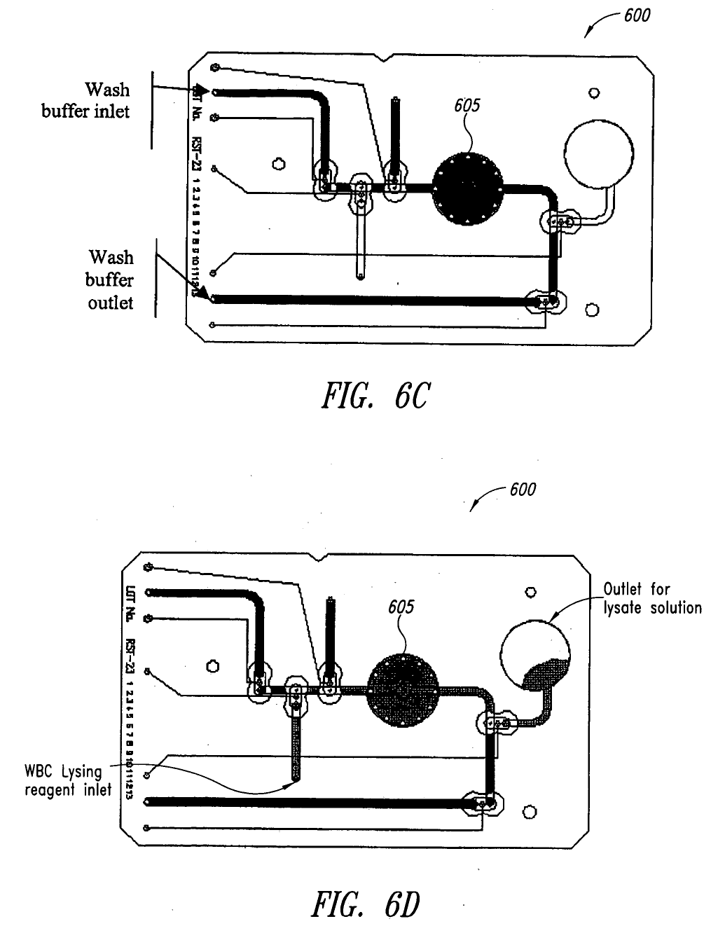

As described with respect to FIGS. 1 and 2, the diverted portion of the whole blood sample containing the labeled cell(s) is captured on a first membrane, which is then washed to remove unwanted cells. A lysis buffer is then passed over the first membrane, lysing the labeled cells and releasing a lysate comprising RNA and DNA. These steps are illustrated in FIGS. 6A-6D, which show a series of cross-sectional views of a microfluidic device 600 illustrating, for example, the operation of a representative sub-circuit for white blood cell capture and lysis in accordance with aspects of the present invention. As shown in FIG. 6A, the sub-circuit comprises a plurality of valves, inlets, outlets and microfluidic channels, in addition to the first membrane 605. FIG. 6B shows the introduction of a whole blood sample into the microfluidic device 600, the capture of white bloods cells on the first membrane 605 and the passage of the depleted whole blood sample (including red blood cells, platelets and plasma) through a waste outlet. FIG. 6C shows the first membrane 605 being washed by a wash buffer. FIG. 6D shows the lysing of the white blood cells captured on the first membrane 605 by a lysis buffer liquid introduced through a lysis buffer microfluidic channel 610 (fluidly connected to the first membrane 605) and the release of the lysate solution for subsequent nucleic acid capture and purification.

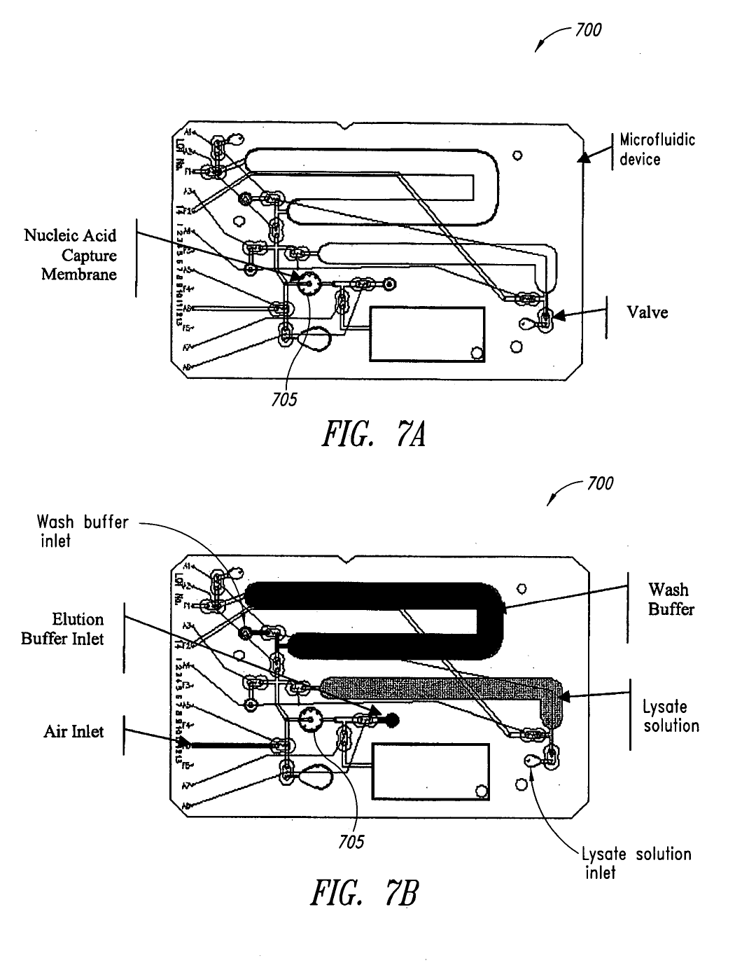

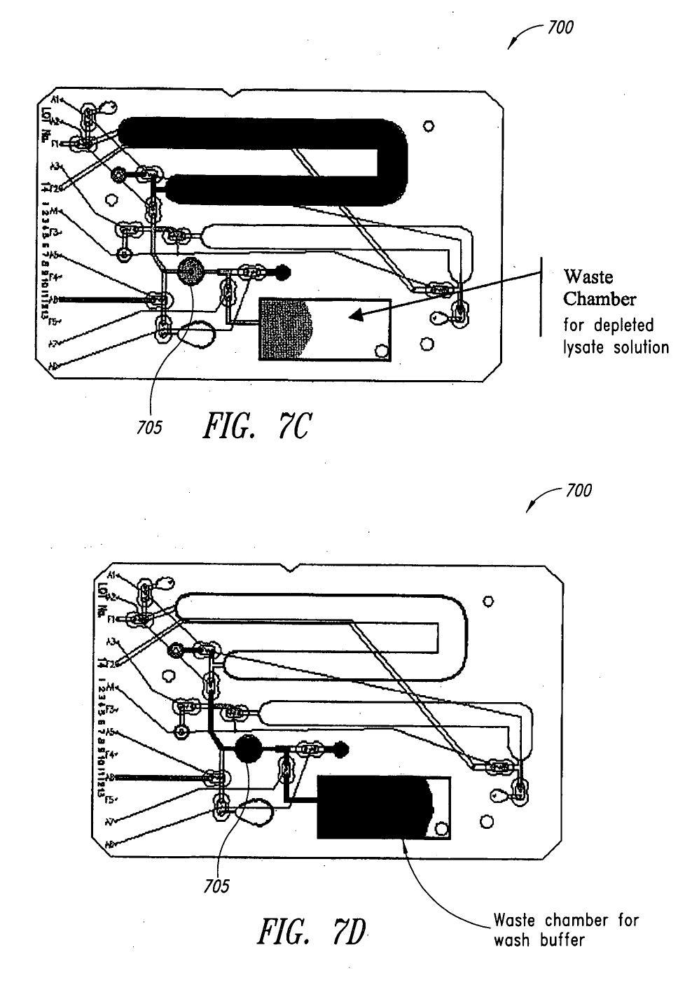

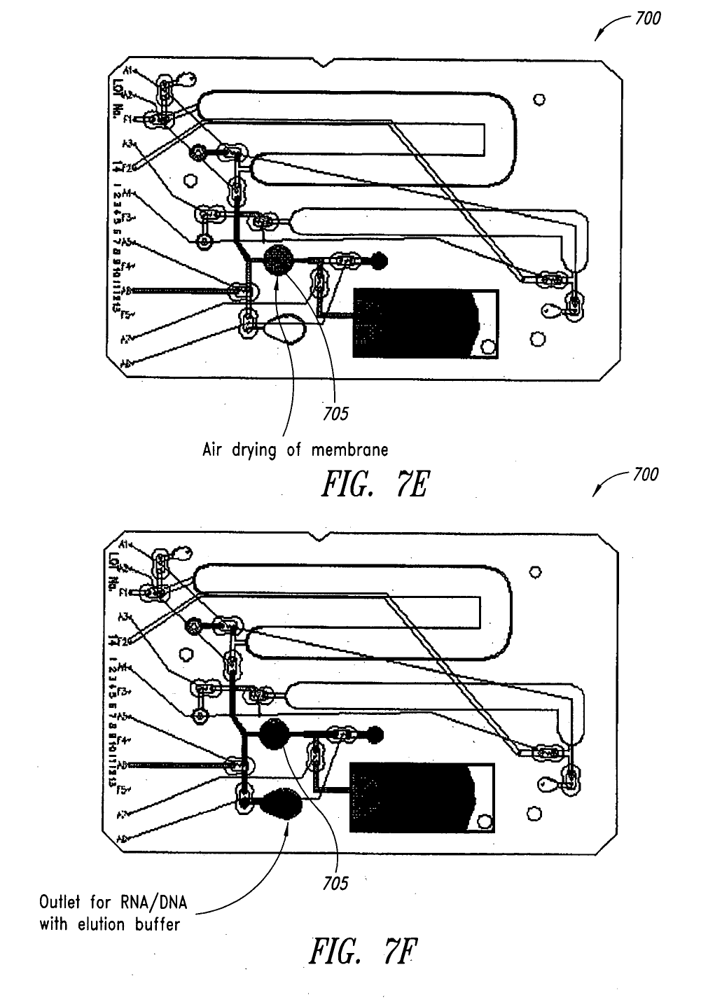

As described above, the resulting lysate solution comprises RNA and DNA, which is then captured on a second membrane. The second membrane is washed and dried to purify the captured RNA/DNA, and the RNA/DNA is then eluted into a PCR amplification chamber. These steps are illustrated in FIGS. 7A-7F, which show a series of cross-sectional views of a microfluidic device 700 illustrating the operation of a representative sub-circuit for nucleic acid capture and purification in accordance with aspects of the present invention. As shown in FIG. 7A, the sub-circuit comprises a plurality of valves, inlets, outlets and microfluidic channels, in addition to the second membrane 705 (i.e., the nucleic acid capture membrane). FIG. 7B shows the introduction of the lysate solution and the wash buffer into the microfluidic device 700. FIG. 7C shows the lysate solution being passed over the second membrane 705—the RNA/DNA is captured on the second membrane 705 and the depleted lysate solution is directed to the waste chamber. FIG. 7D shows the second membrane 705 being washed by the wash buffer. FIG. 7E shows the second membrane 705 being air dried. FIG. 7F shows the release of the RNA/DNA from the second membrane 705 with an elution buffer.

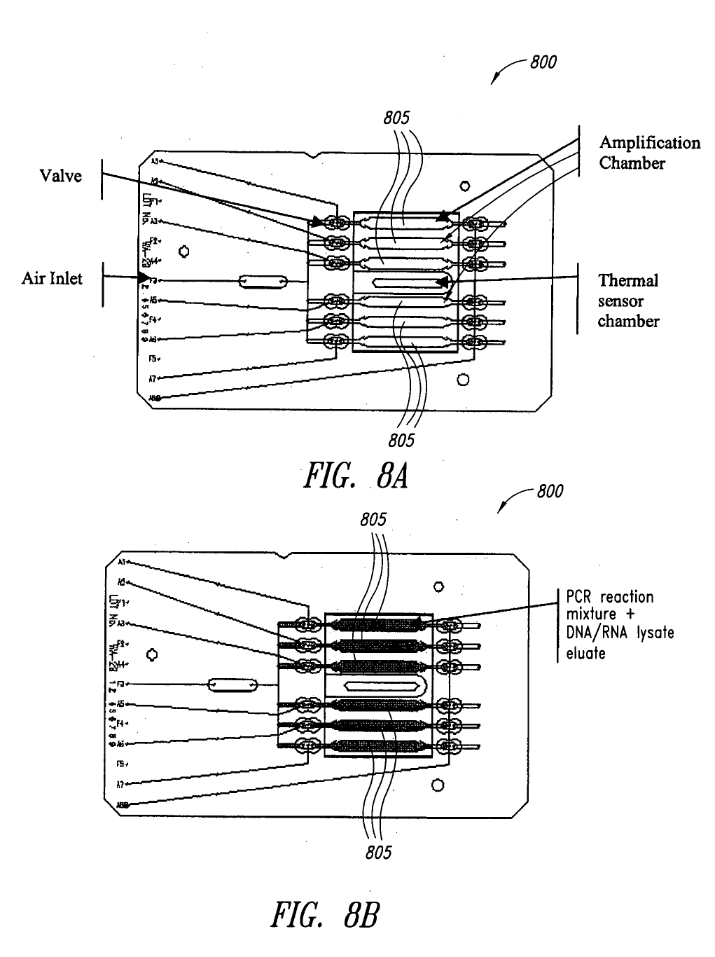

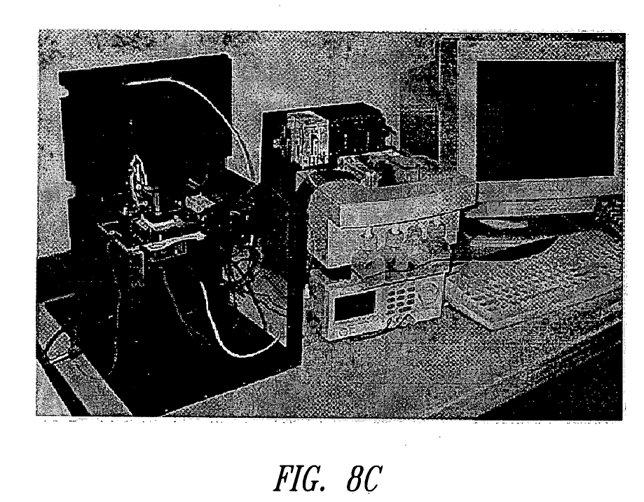

As described with respect to FIGS. 1 and 2, following purification of the RNA/DNA on the second membrane, the purified samples are eluted into one or more PCR amplification chambers wherein quantitative PCR and qRT-PCR analyses may be performed. FIGS. 8A-8B are cross-sectional views of a microfluidic device 800 illustrating the operation of a representative sub-circuit for nucleic acid amplification in accordance with aspects of the present invention. As one of skill in the art will appreciate, the illustrated microfluidic device 800 will be interfaced with both an “off-card” thermal cycler (capable of performing ˜35 cycles) and an “off-card” epi fluorescence detector (capable of quantitatively detecting fluorescence in the amplification chambers). Although not specifically illustrated, the requisite reagents (e.g., probes and primers) for PCR may be (1) introduced into the microfluidic device 800 in liquid form through one or more additional inlets, (2) provided in liquid form in the microfluidic device 800 in one or more blister pouches, or (3) provided in dry form in the microfluidic device 800 by, for example, printing the dry reagents into the PCR amplification chambers 805. FIG. 8C is a photograph of a system incorporating the microfluidic device of FIGS. 8A-8B for nucleic acid amplification in accordance with aspects of the present invention. The system comprises three primary components, namely, a microFlow™ system, a thermal cycler and a power supply.

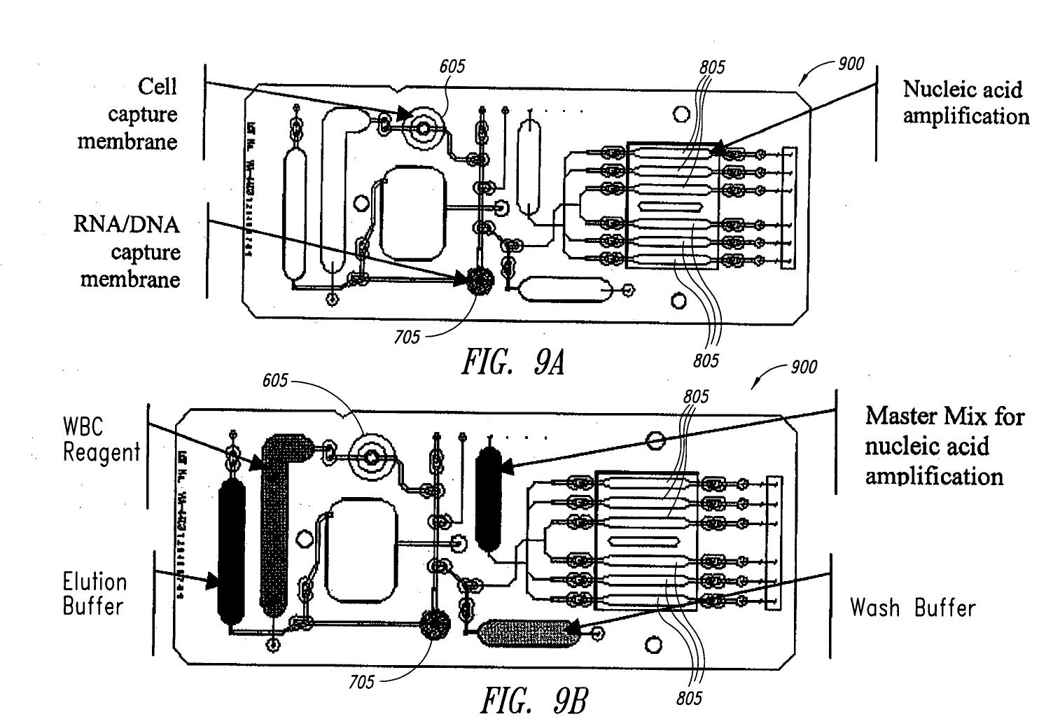

As one of skill in the art will appreciate, the foregoing sub-circuits may be combined in various configurations to produce microfluidic devices for detecting rare cells which integrate and automate sample preparation, cell labeling, cell sorting and enrichment, and DNA/RNA analysis of sorted cells. For example, FIGS. 9A-9B show cross-sectional views of a representative microfluidic device 900 incorporating the sub-circuits of FIGS. 6A-6D (cell capture), 7A-7F (nucleic acid capture) and 8A-8B (PCR amplification) in accordance with aspects of the present invention. As shown, the device of FIGS. 9A-9B integrates cell capture, nucleic acid capture and PCR amplification.

The following examples have been included to illustrate certain embodiments and aspects of the present invention, and should not be construed as limiting in any way.

### EXAMPLES

### Example 1

In the following example, the sample and antibody solutions were moved through the channels of the microfluidic devices by a microFlow™ system, which comprises a controller, pumps (250 μL and 2,300 μL capacity pumps), and a manifold. The microFlow™ system is a commercially available ultra-low-pulse pump system (Micronics, Inc.) with air, vacuum, forward and reverse pumping capabilities controlled by PC based software. In a microfluidic device, fluids can be transported by either air or Fluorinert™ FC-70 (Hampton Research HR2-797). Fluorinert™ FC-70 has a viscosity similar to water, with approximately 75% greater density, and is not miscible with aqueous solutions. In the following examples, Fluorinert™ FC-70 was used to prevent dilution of the sample and antibody solutions during processing.

Lab Card Design

A microfluidic device having the sub-circuits illustrated in FIGS. 3A-3C and 5A-5E was used for the following cell and/or bead counting and sorting experiments. The device comprised a 30 μL sample channel (or loop) for beads and/or cells, an on-card chamber (or reservoir) holding 400 μL of diluted antibody (if used) or PBS (if no antibody used), an on-card thin ribbon formation structure (or sample injector), a labeling channel (or loop), a viewing area, and a sorting slit structure for removal of labeled cells and/or beads. The card was manufactured using laminate prototyping methods (e.g., individual layers were laser cut then laminated to form three-dimensional channels and valves). For the devices used in these experiments, the channels were each 1.5 mm wide and the dwell time in the labeling channel was about 15 seconds.

On-Card Optics

The manifold containing the foregoing lab card was placed on the stage of a Zeiss inverted microscope (model IM35). The card was illuminated with a BlueSky Research 488 nm laser (model FTEC-488-020-SM00). Charge Coupled Device (CCD) cameras (Andor iXon (model DV 877-BI) and Watec (model LCL-902C, monochrome)) were used to view the lab card through the microscope. The Watec camera has traditional video output and video from this camera was captured using a National Instruments video capture card (model IMAQ PCI-1409). The data was collected in movie format, which allowed for analysis on-the-fly or could be saved for further analysis at a later time. The analysis portion used National Instruments LabVIEW® (Version 6i) with the Vision add-on (IMAQ Vision for LabVIEW). This software comes with “blob” analysis, which can be configure to recognize bright spots in an area of interest and is used to count beads or cells as they pass through the laser spot.

Counting speed was determined by the camera frame speed and light sensitivity. The sensitivity also was determined by the brightness of the labeled cells or beads. For these experiments, only brightly labeled cells or beads were used. Speed can be increased if the camera reads only a small portion of the field of view. The Watec camera was fixed at 30 frames per second (FPS) while the iXon FPS was determined by the configuration number of pixels per line and number of lines as well as readback speed. Maximum FPS for the iXon was 200 FPS. However, faster readback decreases the signal-to-noise ratio, so speed and resolution were a trade-off.

Fluorescent Bead Controls

All beads used for these experiments were obtained from Polysciences, Inc. Initial visualization used very bright Fluoresbrite® Yellow Green Microspheres. Both 3 μm (calibration grade #17147) and 10 μm (#18140) beads were mixed. These beads are deeply dyed, with nearly the entire bead labeled. Next, a medium bright Flow Check FITC 6 μm bead (#24253) was visualized. These beads are not deeply dyed (typically only the outer 10%) and show a ghostlike appearance. Finally, PolyComp beads coated with anti-IgG (#24312) were tagged with the CD4 antibody either on or off card. These beads have both bright and medium bright fluorescence levels. Bead size is unspecified but appears to be about ˜6 μm.

CD4 White Blood Cell Labeling

For feasibility testing, CD4 was utilized in order to begin with higher cell counts using well established reagents and labeling protocols. The CD4 antibody used was BD Biosciences Pharmingen #557695 AlexaFluor® 488 conjugated mouse anti-human CD4. This CD4 antibody is known to stain approximately 15% of the white blood cells in an average blood sample. The CD4 antibodies used were tagged with AlexFluor 488, a dye with similar response to fluorescein conjugates, but more photostable. When illuminated with light of wavelength 488 nm (blue), AlexFluor 488 emits with a wavelength of ˜520 nm (green). The Zeiss microscope was outfitted with a filter set that limits transmission to the camera of fluorescent light only, eliminating scatter. No excitation filter was used due to the use of the 488 nm laser. Either a 510 nm 20 db bandpass filter (Chroma Technology Corp #D510/20x—part of filter set 31040) or a 520 nm 40 db bandpass filter (Omega #XF3003) with a beamsplitter (Chroma Technology Corp #505dclp—part of set CZ 716) was used.

The recommended protocol for the CD4 antibodies specifies use of 5 μL antibody reagent to 100 μL whole blood. For on-card labeling tests, the 5 μL of antibody was diluted in 200 μL PBS in order to provide the sheath volume needed to create the labeling ribbon.

A comparison test was run with white blood cells prepared off-card using standard accepted practices. Whole blood was mixed with EDTA and stored at 4° C. The protocol used 100 μL whole blood and lysed red blood cells with 1.4 mL ammonium chloride. Cells were then washed with PBS and stored at 4° C. until used. When used, cells were re-suspended in 100 μL PBS to obtain concentration similar to whole blood.

Fluorescent Bead Counts on Card

Various types of beads, both fluorescent and functionalized, were loaded into the 30 μL sample loop of the lab card. The antibody reservoir on card was filled with PBS alone (for non-labeled sample) or CD4 mABS diluted with PBS (for on-card labeling of functionalized beads). The sample was pushed with Fluorinert and the antibody reservoir fluid was pushed with PBS, if not labeling on card, or Fluorinert, for on-card labeling. Fluorinert was used in order to prevent dilution of the antibody spiked PBS. Various sample and sheath rates were tested. Good labeling occurred with a 10:1 antibody spiked sheath:sample flow rate ratio. The sheath flow rate of 1.0 allowed for a slow CCD to obtain a good view of each bead or cell. At this flow rate, a sample of 10 μL would take about 2 minutes to run. The sampling portion of the test took ˜15 seconds. Fluorescence bead counting was successful with a very accurate correlation to both expected and measured counts, as shown in Table 1.

TABLE 1

| | | | Sample | Label | | | | |
|---|---|---|---|---|---|---|---|---|
| | Particle | | Flow | Flow | # | Expected | Observed | Ratio |
| Sample | Size | Labeled | Rate | Rate | Frames/Frames | Number | Number | Observed:Expected |
| Type | (μm) | on-card | (μL/sec) | (μL/sec) | per Sec | of Beads* | of Beads* | Beads |
| Fluoresbrite | 3 | No | 0.025 | 1.0 | 100/33 | 37 | 36 | 0.97 |
| (mixed | — | | | | | — | — | — |
| sizes) | 10 | | | | | 2.3 | 4 | 1.74 |
| FITC | 6 | No | 0.1 | 0.5 | 100/77 | 42.8 | 48 | 1.12 |
| PolyComp | ˜6 | No | 0.1 | 1.0 | 100/30 | 22 | 20 | 0.91 |
| PolyComp | ˜6 | 1 μl CD4 | 0.1 | 1.0 | 30/30 | 6.6 | 3 | 0.45 |
| | | mAbs: 40 μl | | | | | | |
| | | PBS | | | | | | |

*(50 × 75 μm beam spot)

Fluorescent WBC Counts on Card

WBC counts were calculated using average expected values. All blood samples were from the same individual. Table 2 presents a summary of the test results. Since only a few cells are expected to be present in the small area of the channel illuminated, the calculated ratios can change with the presence or absence of a single cell. Longer run times and samples from various donors will be tested to provide more statistical significance.

TABLE 2

| | | Sample | Label | # | Expected | Observed | |
|---|---|---|---|---|---|---|---|
| | | Flow | Flow | Frames/ | Number of | Number of | Ratio |
| | Labeled on- | Rate | Rate | Frames | Labeled | Labeled | Observed:Expected |
| Sample Type | card | (μl/sec) | (μl/sec) | per Sec | Cells* | Cells* | Cells |
| CD4+ Labeled | No | 0.05 | 0.55 | 100/91 | 1.1 | 2 | 1.85 |
| White Blood | | | | | | | |
| Cells | | | | | | | |
| White Blood | 1 μl CD4 | 0.1 | 1.0 | 50/30 | 3.3 | 4 | 1.21 |
| Cells | mAbs: 40 μl PBS | | | | | | |

*(50 × 75 μm beam spot)

Antibody Labeling on Card

Table 3 details the time required for the various steps used to label cells prior to sorting. As shown, the normal protocol takes over an hour while the on-card process in completed within 30 seconds. The volume of reagents was also reduced and the waste was safely contained on-card.

TABLE 3

| | Standard Assay | |
|---|---|---|
| Assay Step | Conditions | On-Card Assay |
| Whole Blood Sample | 100 μL | 12 μL |
| Dilution with PBS | 400 μL | |
| Mabs (Labeled Antibody) | 5 μL | 0.6 μL (non-optimized) |
| Dilution with PBS | | 258 μL |
| Incubate | 20-30 minutes @ | 20 seconds at ambient |
| | 4° C. | temp |
| Centrifuge | 5 minutes | |
| Remove supernatant | 30 seconds | |
| Add lysing solution | 1.4 mL | 500 μL non-optimized |
| Incubate at room temp | 3-5 minutes | 20 seconds |
| Centrifuge | | |
| Remove supernatant | 30 seconds | |
| Dilutions with PBS | 600 μL | |
| Remove supernatant | 30 seconds | |
| Dilution with PBS | 400 μL | |
| Cytometric Measurement | 2-3 minutes | 2-3 minutes |

Sorting/Fluorescence Gating

For feasibility testing, the goal was to demonstrate manual sorting of beads. Beads used either alone or mixed with WBCs were run through the system and captured. The sorting volume displaced within the plastic card was defined by the slit width (25 μm), slit length (1,500 μm), and slit depth (150 μm). A 2300 μL capacity pump was used to aspirate fluid at 30 μL/sec. The pump displaced about 1-2 μL of fluid and was chosen for the rapid flow rate rather than for small displacement volume. For cell or bead sorting, the sample flow rate was 0.1 μL/sec and the antibody labeling solution flow rate was 1 μL/sec. The highest frequency of sorting was measured at 0.91 seconds per sorting pulse, using the pumps on the microFlow system to sort cells.

The thin ribbon cell sorter appears to be a very feasible method of rare cell sorting from whole blood cells. The labeled cells were visualized and their velocity recorded for purposes of determining when to sort a cell. The sorting volume was small and should effectively reduce the number of cells to be analyzed. Cell displacement from a moving stream works well and with some optimization should be able to sample a small volume within the moving stream.

### Example 2

Lab Card and Microfluidic Circuitry

A microfluidic device having the sub-circuits illustrated in FIGS. 7A-7F was used to evaluate automated liquid handling steps for RNA extraction. The device comprised a 700 μL wash solution chamber, a 150 μL elution solution chamber, a 250 μL lysate/binding solution chamber, and a silica membrane assembly. The silica membrane assembly comprised two circular glass fiber filter type D membranes (GF/D, 8 mm diameter discs, Whatman). The fluidic circuitry used on-card valving to control fluid paths and a simple vacuum to deliver and draw solutions through the GF/D membranes Additionally, a small volume pump (˜150 μL) was used to deliver the elution solution. After loading the device with appropriate solutions, the device automated the following steps: (1) white blood cells in lysate solution were pulled across the silica membrane by vacuum; (2) nucleic acid from the cells were bound to the membrane under the lysis conditions used; (3) a wash solution was pulled across the membrane to remove cellular debris; (4) the membrane was dried by pulling air through the channels; (5) an elution solution was pumped over the membrane; and (6) the RNA in eluant was taken off-card via pipet for analysis. The foregoing steps were completed in less than 5 minutes.

RNA Control

The RNA control for the BCR-ABL fusion transcript was acquired from total human RNA isolated from the K562 cell line. The K562 cell line, which was derived from chronic myelogenous leukemia (CML) cells isolated from peripheral blood, was used as the source of the BCR-ABL fusion transcript being investigated. White blood cells were isolated from whole blood using a red blood cell (RBC) lysis solution (Gentra). The collected WBCs were stabilized using RNAlater® reagent (QIAGEN) and were stored at −20° C. until just prior to lysis.

Comparative RNA Isolation Methods

The two kits used to assess the performance of the foregoing lab card were Rneasy® (QIAGEN) and MagnaPure® (Roche). The RNeasy kit uses silica-based micro-centrifuge spin columns along with proprietary lysis, binding and wash chemistries to isolate total RNA from cellular lysate. As an alternative, the MagnaPure kit, which was the recommended method of choice for the LightCycler® (Roche) platform, uses magnetic beads with tethered oligonucleotide probes along with proprietary lysis, binding and wash chemistries to isolate messenger RNA.

Quantitative Measure of RNA Yield

A LightCyler® RT-PCR quantification kit (Roche) was used for relative quantification of BCR-ABL fusion transcripts. The kit contained reagents to perform quantitative RT-PCR of both BCR-ABL and Glucose-6-Phosphate Dehydrogenase (G6PDH) gene transcripts. Reverse transcription and PCR were performed in two separate steps. The G6PDH housekeeping target served as both a control for RT-PCR performance and as a reference for relative quantification of transcript expression.

Platinum Quantitative RT-PCR Thermoscript One-Step System (Invitrogen) was the reagent kit used for single-step reverse transcription PCR. Using this kit, single-step endpoint amplification of RNA transcripts was performed on both a conventional thermalcycler (MJ Research). Primers were designed for both G6PDH and BCR-ABL using a commercially available primer design tool (Oligo6; Molecular Biology Insights, Inc.).

Verify On-Card RNA Extraction

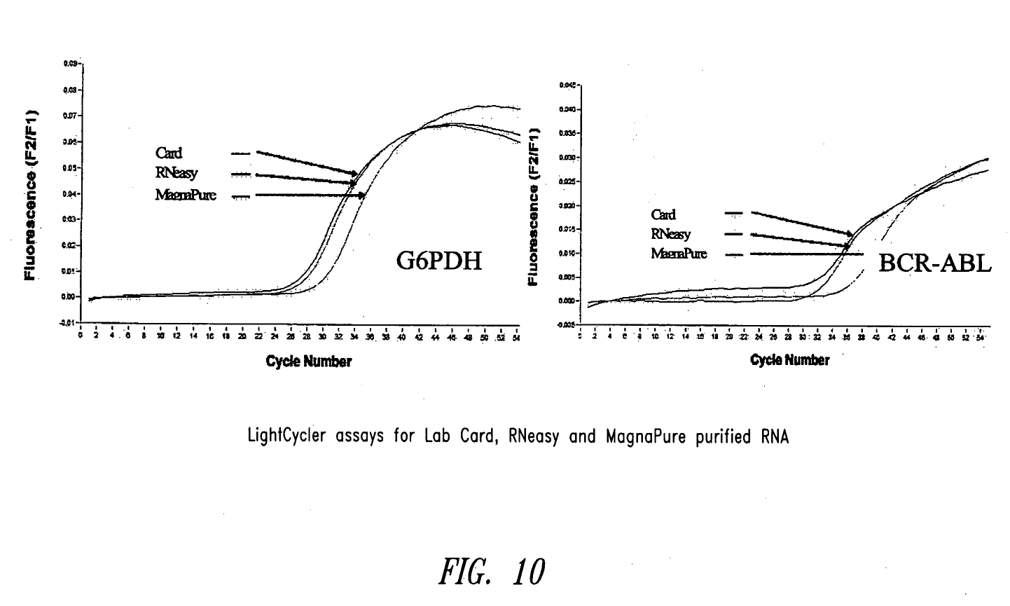

Initial functional validation of the proposed lab card solutions (i.e., binding solution, wash solution and elution solution) were done using the glass fiber based purification columns provided in the RNeasy performance standard. Approximately 1×10⁶ WBCs were processed using both the lab card and performance standard chemistries. The resultant purified RNA samples were assayed by LightCycler to determine if both sets of chemistries yielded similar relative quantities of G6PDH transcript. As indicated by the data shown in FIG. 10, the proposed microfluidic card solutions had a slightly lower crossing point (25.3) than the control RNeasy solutions (27.1) and therefore should be considered at least as good as the control with respect the quantity and quality of RNA recovered.

The lab card was then validated using the described card-compatible solutions and was compared to two established standard methods for RNA purification: RNeasy and MagnaPure. Approximately 1×10⁶ WBCs spiked with 250 ng of K562 RNA (Cell-RNA amounts per 10 μL; K562 RNA was added to provide BCR-ABL transcript) were processed using each of the purification methods. Accepted standard methods were performed according to manufacturers' instructions. The resulting samples were then assayed by LightCycler to measure both G6PDH and BCR-ABL RNA transcript. As shown in FIG. 10, the RNA processed on the lab card had crossing points of 27.8 and 30.8 for G6PDH and BCR-ABL, respectively. Similarly, the RNeasy control had crossing points of 27.3 and 30.4 for G6PDH and BCR-ABL, respectfully. In contrast, with crossing points of 30.1 and 36.2 for G6PDH and BCR-ABL, respectfully, the MagnaPure control kit yielded a diminished quantity and/or quality of RNA relative to that observed for either the lab card or RNeasy method. As such, the lab card method for total RNA isolation was demonstrated to be at least as good as the RNeasy performance standard with respect to the quantity and quality of RNA recovered, and both methods significantly outperformed the MagnaPure method.

On-Card RNA Limit of Detection

With on-card validation complete, experiments were expanded to evaluate the limit of detection of BCR-ABL mRNA transcripts. Similar to the validation experiment described above, the lab card described above was compared to both the RNeasy and MagnaPure standard methods for these experiments.

A stock of approximately 1×10⁶ WBCs spiked with 250 ng of K562 RNA (Cell-RNA amounts per 10 μL; K562 RNA) added to provide BCR-ABL transcript) was serially diluted (1:2) with water to produce five dilutions, which, along with the undiluted stock, were processed using each of the purification methods. The dilutions were prepared such that at the lowest dilution level, the K562 total RNA would be assayed at a level representative of a reasonably low cell equivalent number. For this experiment, the lowest dilution of K562 total RNA assayed by LightCyler was 0.2 ng/assay, which, when using the value of 1 ng total RNA per 50 K562 cells provided in the LightCycler manual, was equivalent to 10 total K562 cells. As before, accepted standard methods were performed according to manufacturers' instructions.

The purified dilution series were first assayed by LightCycler to measure both G6PDH and BCR-ABL RNA transcript limits of detection. Similar to the initial validation experiment described, the plastic purification subcircuit and RNeasy standard yielded total RNA comparable in performance when evaluated by both G6PDH and BCR-ABL LightCycler assay, whereas the MagnaPure method yielded mRNA with clearly diminished quantity and/or quality, as shown in Table 4.

TABLE 4
G6PDH ASSAY
Equivalent WBCs/RNA used per LightCyler Reaction*

| | | | Control in | | | |
|---|---|---|---|---|---|---|
| | WBC's | K562 Total RNA | vitro transcript | Control mRNA | Crossing | Endpoint |
| Sample Description | (# Cells) | (ng RNA) | (fg RNA) | (ng mRNA) | Point | Amlification** |
| G6PDH RNA I | — | — | 50 | — | 19.89 | (+) |
| G6PDH RNA II | — | — | 2.5 | — | 23.73 | (+) |
| G6PDH RNA III | — | — | 0.05 | — | 28.03 | (+) |
| t(9; 22) mRNA(+) Control | — | — | — | 1.25 | 24.60 | (+) |
| K562 RNA | — | 6.25 | — | — | 28.00 | (+) |
| Water | — | — | — | — | — | (−) |
| Card Dilution 1 | 25000 | 6.25 | — | — | 28.15 | (+) |
| Card Dilution 2 | 12500 | 3.13 | — | — | 27.83 | (+) |
| Card Dilution 3 | 6250 | 1.56 | — | — | 28.00 | (+) |
| Card Dilution 4 | 3125 | 0.78 | — | — | 29.17 | (+) |
| Card Dilution 5 | 1563 | 0.39 | — | — | 29.81 | (+) |
| Card Dilution 6 | 781 | 0.20 | — | — | — | (−) |
| Card Negative Control | 0 | 0.00 | — | — | — | (−) |
| RNeasy Dilution 1 | 25000 | 6.25 | — | — | 28.53 | (+) |
| RNeasy Dilution 2 | 12500 | 3.13 | — | — | 27.26 | (+) |
| RNeasy Dilution 3 | 6250 | 1.56 | — | — | 28.83 | (+) |
| RNeasy Dilution 4 | 3125 | 0.78 | — | — | 30.00 | (+) |
| RNeasy Dilution 5 | 1563 | 0.39 | — | — | 31.06 | (+) |
| RNeasy Dilution 6 | 781 | 0.20 | — | — | 32.14 | (+) |
| RNeasy Negative Control | 0 | 0.00 | — | — | — | (−) |
| MagnaPure Dilution 1 | 25000 | 6.25 | — | — | 31.98 | (+) |
| MegnaPure Dilution 2 | 12500 | 3.13 | — | — | 33.29 | (+) |
| MagnaPure Dilution 3 | 6250 | 1.56 | — | — | 30.10 | (+) |
| MagnaPure Dilution 4 | 3125 | 0.78 | — | — | 33.21 | (+) |
| MagnaPure Dilution 5 | 1563 | 0.39 | — | — | — | (+) |
| Megnapure Dilution 6 | 781 | 0.20 | — | — | — | (+) |
| MagnaPure Negative Control) | 0 | 0.00 | — | — | — | (−) |

BCR-ABL ASSAY
Equivalent WBCs/RNA used per LightCyler Reaction*

| | WBC's | K562 Total RNA | vitro transcript | Control mRNA | Crossing | Endpoint |
|---|---|---|---|---|---|---|
| SAMPLE DESCRIPTION | (# Cells) | (ng RNA) | (fg RNA) | (ng mRNA) | Point | Amlification** |
| G6PDH RNA I | — | — | 50 | — | — | (−) |
| G6PDH RNA II | — | — | 2.5 | — | — | (−) |
| G6PDH RNA III | — | — | 0.05 | — | — | (−) |
| t(9; 22) mRNA(+) Control | — | — | — | 1.25 | 25.69 | (+) |
| K562 RNA | — | 6.25 | — | — | 29.18 | (+) |
| Water | — | — | — | — | — | (−) |
| 1 Card | 25000 | 6.25 | — | — | 30.20 | (+) |
| 2 Card | 12500 | 3.13 | — | — | 31.78 | (+) |
| 3 Card | 6250 | 1.56 | — | — | 30.84 | (+) |
| 4 Card | 3125 | 0.78 | — | — | 31.78 | (+) |
| 5 Card | 1563 | 0.39 | — | — | 32.65 | (+) |
| 6 Card | 781 | 0.20 | — | — | — | (−) |
| 7 Card | 0 | 0.00 | — | — | — | (−) |
| 1Q | 25000 | 6.25 | — | — | 31.92 | (+) |
| 2Q | 12500 | 3.13 | — | — | 30.38 | (+) |
| 3Q | 6250 | 1.56 | — | — | 32.01 | (+) |
| 4Q | 3125 | 0.78 | — | — | 33.84 | (+) |
| 5Q | 1563 | 0.39 | — | — | 34.25 | (+) |
| 6Q | 781 | 0.20 | — | — | 36.75 | (+) |
| 7Q | 0 | 0.00 | — | — | — | (−) |
| 1 Mag | 25000 | 6.25 | — | — | 36.23 | (−) |
| 2 Mag | 12500 | 3.13 | — | — | — | (−) |
| 3 Mag | 6250 | 1.56 | — | — | — | (−) |
| 4 Mag | 3125 | 0.78 | — | — | — | (−) |
| 5 Mag | 1563 | 0.39 | — | — | — | (−) |
| 6 Meg | 781 | 0.20 | — | — | — | (−) |
| 7 Mag | 0 | 0.00 | — | — | — | (−) |

* 1/40th (2.5 ul) from each purification was used per LightCyler reaction. The indicated equivalents is the fractional amount of cells/RNA represented by the volume of RNA used per LightCycler reaction.
**Enpoint amplification on MJ thermalcycler. Reaction products visualized on gel: (+) = expected band observed: (−) = no band

Concentrating analysis on the BCR-ABL LightCycler assay results, the RNeasy method successfully amplified all six dilutions, thus achieving sensitivity down to at least 10 cell equivalents per assay. The lab card successfully amplified the first five sample dilutions to achieve sensitivity down to at least 20 cells and, consistent with all data produced thus far, the MagnaPure method successfully amplified only the highest concentration sample, which was equivalent to over 300 K562 cells. The average crossing point for the both the RNeasy control and the microfluidic purification card was 32.5 and 31.5, respectively, thus confirming that both methods yield RNA of similar quality. In contrast, the crossing point for the only MagnaPure reaction that amplified was 36.2.

Experimentation was done on the same dilution series described above using a single-step reverse transcription PCR kit (Invitrogen) amplified using a conventional thermalcycler (MJ Research). Amplification products were resolved by agarose gel electrophoresis and were visualized by ethidium bromide staining (data not shown). Positive endpoint amplification was represented by a (+) for visualization of expected band and (−) for no observed band as shown in Table 4. With few exceptions for some of the MagnaPure purified RNA, the described approach yielded the same endpoint results as those observed for the LightCycler assay.

From the foregoing, and as set forth previously, it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A microfluidic device for detecting rare cells, comprising:
means for introducing a biological sample into the microfluidic device, wherein the biological sample comprises one or more labeled cells;
means for sheathing the biological sample with a buffer liquid to form a thin ribbon of the biological sample;
means for facilitating the detection of the labeled cells in the biological sample;
means for separating the labeled cells from the biological sample;
means for lysing the labeled cells;
means for collecting RNA and DNA released from the lysed labeled cells; and
means for performing quantitative PCR analysis of the collected RNA and DNA.

2. The microfluidic device of claim 1 wherein the means for introducing a biological sample into the microfluidic device comprises a sample inlet port fluidly connected to a sample inlet microfluidic channel.

3. The microfluidic device of claim 1 wherein the means for sheathing the biological sample with a buffer liquid to form a thin ribbon of the biological sample comprises a thin ribbon sheath flow assembly.

4. The microfluidic device of claim 3 wherein the thin ribbon sheath flow assembly comprises a sample microfluidic channel, a first sheath liquid microfluidic channel and a second sheath liquid microfluidic channel, wherein the first and second sheath liquid microfluidic channels are positioned on opposing sides of, and fluidly converge with, the sample microfluidic channel.

5. The microfluidic device of claim 1 wherein the means for facilitating the detection of the labeled cells in the biological sample comprises an optical viewing window positioned over a portion of a sheathed sample microfluidic channel.

6. The microfluidic device of claim 1 wherein the means for separating the labeled cells from the biological sample comprises a cell sorting slit structure.

7. The microfluidic device of claim 1 wherein the means for separating the labeled cells from the biological sample comprises a cell sorting flexible film structure comprising a flexible film membrane, the flexible film membrane being deformable into a sheathed sample microfluidic channel upon the application of pneumatic pressure.

8. The microfluidic device of claim 1 wherein the means for separating the labeled cells from the biological sample comprises an electromagnetically actuated valve.

9. The microfluidic device of claim 8 wherein the electromagnetically actuated valve comprises a metal foil.

10. The microfluidic device of claim 1 wherein the means for lysing the labeled cells comprises a first membrane, adapted to capture the labeled cells, and a lysis buffer microfluidic channel fluidly connected to the first membrane.

11. The microfluidic device of claim 10 wherein the first membrane is a polybutylene terephthalate membrane.

12. The microfluidic device of claim 1 wherein the means for lysing the labeled cells comprises a lysis buffer sheath flow assembly.

13. The microfluidic device of claim 12 wherein the lysis buffer sheath flow assembly comprises a sorted cell microfluidic channel, a first lysis buffer microfluidic channel and a second lysis buffer microfluidic channel, wherein the first and second lysis buffer microfluidic channels are positioned on opposing sides of, and fluidly converge with, the sorted cell microfluidic channel.

14. The microfluidic device of claim 1 wherein the means for collecting RNA and DNA released from the lysed labeled cells comprises a second membrane, adapted to capture the released RNA and DNA.

15. The microfluidic device of claim 14 wherein the second membrane comprises glass.

16. The microfluidic device of claim 14 wherein the second membrane comprises silicate.

17. The microfluidic device of claim 1 wherein the means for performing quantitative PCR analysis of the collected RNA and DNA comprises a PCR amplification chamber.

18. The microfluidic device of claim 17 wherein the PCR amplification chamber comprises PCR probe and primer reagents.

19. The microfluidic device of claim 1 wherein the biological sample is a blood sample.

20. A microfluidic device for detecting rare cells, comprising:
means for introducing a biological sample into the microfluidic device;
means for sheathing the biological sample with a labeling buffer liquid to form a thin ribbon of the biological sample and label one or more cells in the biological sample;
means for facilitating the detection of the labeled cells in the biological sample;
means for separating the labeled cells from the biological sample;
means for lysing the labeled cells;
means for collecting RNA and DNA released from the lysed labeled cells; and
means for performing quantitative PCR analysis of the collected RNA and DNA.

21. The microfluidic device of claim 20 wherein the means for introducing a biological sample into the microfluidic device comprises a sample inlet port fluidly connected to a sample inlet microfluidic channel.

22. The microfluidic device of claim 20 wherein the means for sheathing the biological sample with a labeling buffer liquid to form a thin ribbon of the biological sample and label one or more cells in the biological sample comprises a thin ribbon labeling sheath flow assembly.

23. The microfluidic device of claim 22 wherein the thin ribbon labeling sheath flow assembly comprises a sample microfluidic channel, a first labeling sheath liquid microfluidic channel and a second labeling sheath liquid microfluidic channel, wherein the first and second labeling sheath liquid microfluidic channels are positioned on opposing sides of, and fluidly converge with, the sample microfluidic channel.

24. The microfluidic device of claim 20 wherein the means for facilitating the detection of the labeled cells in the biological sample comprises an optical viewing window positioned over a portion of a sheathed sample microfluidic channel.

25. The microfluidic device of claim 20 wherein the means for separating the labeled cells from the biological sample comprises a cell sorting slit structure.

26. The microfluidic device of claim 20 wherein the means for separating the labeled cells from the biological sample comprises a cell sorting flexible film structure comprising a flexible film membrane, the flexible film membrane being deformable into a sheathed sample microfluidic channel upon the application of pneumatic pressure.

27. The microfluidic device of claim 20 wherein the means for separating the labeled cells from the biological sample comprises an electromagnetically actuated valve.

28. The microfluidic device of claim 27 wherein the electromagnetically actuated valve comprises a metal foil.

29. The microfluidic device of claim 20 wherein the means for lysing the labeled cells comprises a first membrane, adapted to capture the labeled cells, and a lysis buffer microfluidic channel fluidly connected to the first membrane.

30. The microfluidic device of claim 29 wherein the first membrane is a polybutylene terephthalate membrane.

31. The microfluidic device of claim 20 wherein the means for lysing the labeled cells comprises a lysis buffer sheath flow assembly.

32. The microfluidic device of claim 31 wherein the lysis buffer sheath flow assembly comprises a sorted cell microfluidic channel, a first lysis buffer microfluidic channel and a second lysis buffer microfluidic channel, wherein the first and second lysis buffer microfluidic channels are positioned on opposing sides of, and fluidly converge with, the sorted cell microfluidic channel.

33. The microfluidic device of claim 20 wherein the means for collecting RNA and DNA released from the lysed labeled cells comprises a second membrane, adapted to capture the released RNA and DNA.

34. The microfluidic device of claim 33 wherein the second membrane comprises glass.

35. The microfluidic device of claim 33 wherein the second membrane comprises silicate.

36. The microfluidic device of claim 20 wherein the means for performing quantitative PCR analysis of the collected RNA and DNA comprises a PCR amplification chamber.

37. The microfluidic device of claim 36 wherein the PCR amplification chamber comprises PCR probe and primer reagents.

38. The microfluidic device of claim 20 wherein the biological sample is a blood sample.
